(19) United States
(12) Patent Application Publication
(10) Pub. No.: US 2018/0235916 A1
(43) Pub. Date: Aug. 23, 2018

(54) ENHANCING BETA CELL REPLICATION AND/OR SURVIVAL

(71) Applicant: The Regents of the University of California, Oakland, CA (US)

(72) Inventors: Daniel L. Kaufman, Los Angeles, CA (US); Jide Tian, Los Angeles, CA (US)

(21) Appl. No.: 15/761,795

(22) PCT Filed: Oct. 12, 2016
(86) PCT No.: PCT/US2016/056528

§ 371 Date: Mar. 20, 2018

Related U.S. Application Data
(60) Provisional application No. 62/279,908, filed on Jan. 18, 2016.
(60) Provisional application No. 62/241,566, filed on Oct. 14, 2015.

(51) Int. Cl. A61K 31/197; A61K 31/5517; A61K 31/5513

(52) U.S. Cl. CPC A61K 31/197; A61K 31/5513; A61K 31/5517

(57) ABSTRACT

In certain embodiments methods of promoting the replication, and/or survival, and/or function of beta cells in mammal are provided where the methods comprise administering to a mammal a GABA receptor activating ligand in conjunction with a GABA_{A} receptor positive allosteric modulator (PAM) where the GABA receptor activating ligand is used at a lower dosage than would be used to achieve the same effect on beta cell replication, and/or survival, and/or function when used alone and/or said positive allosteric modulator is used at a subtherapeutic dosage.

### CROSS-REFERENCE TO RELATED APPLICATIONS

This application claims benefit of and priority to U.S. Ser. No. 62/241,566, filed on Oct. 14, 2015, and to U.S. Ser. No. 62/279,908, filed on Jan. 18, 2016, both of which are incorporated herein by reference in their entirety for all purposes.

### STATEMENT OF GOVERNMENTAL SUPPORT

This invention was made with government support under Grant No. DK092480 awarded by the National Institutes of Health. The Government has certain rights in this invention.

### BACKGROUND

Beta cells (β-cells) are a type of pancreatic cell located in the islets of Langerhans in the pancreas which make and secrete insulin, a hormone that controls the level of glucose in the blood.

The pancreas serves two major functions: (i) the production of digestive enzymes, that are secreted by exocrine acinar cells and routed to the intestine by a branched ductal network; and (ii) the regulation of blood sugar, which is achieved by endocrine cells of the islets of Langerhans. Several separate endocrine cell types comprise the islet. Pancreatic β cells, also referred to as β-cells or “beta cells”, are the most prominent representing about 50-80% of the total depending on species. B-cells produce a number of polypeptides including insulin, a hormone that controls the level of glucose in the blood, C-peptide, a byproduct of insulin production, that helps to prevent neuropathy and other symptoms of diabetes related to vascular deterioration, and amylin, also known as islet amyloid polypeptide (IAP, or IAPP), which functions as part of the endocrine pancreas and contributes to glycemic control. Glucagon-producing α-cells are the next most-common cell type. The remaining islet cells, each comprising a small minority of the total, include δ-cells, which produce somatostatin, PP cells, which produce pancreatic polypeptide; and ε-cells, that produce ghrelin.

Impaired function and/or diminished numbers of beta cells are implicated in metabolic diseases including diabetes, obesity, and other disorders.

### SUMMARY

It was discovered that GABA can inhibit autoimmune responses in different models of T cell-mediated autoimmune diseases, enhance Treg responses, inhibit beta-cell apoptosis and promote mouse beta-cell replication. Extending these findings, it was discovered that activation of both GABA_{A}-receptors (GABA_{A}-Rs) and GABA_{B}-Rs can promote mouse beta-cell replication and human beta-cell replication as well. GABA however, has a relatively low affinity for its receptors, a fast off-rate and a short half-life in vivo, which may necessitate that patients take several grams of GABA a few times a day, for therapeutic efficacy, which is somewhat cumbersome and reduces patient compliance.

It was a surprising discovery that GABA receptor activating ligands in combination with GABA_{A}-R positive allosteric modulators (PAMS) can promote the replication, and/or survival, and/or function (e.g., insulin production, sensitivity to blood sugar, etc.) of beta cells in mammal. Moreover, it was particularly surprising that the combination of a GABA receptor activating ligand and a PAM is synergistic in these effects on beta cells.

Various embodiments contemplated herein may include, but need not be limited to, one or more of the following:

Embodiment 1: A method of promoting the replication, growth, and/or survival, and/or function of beta cells in mammal, said method comprising administering to said mammal a GABA_{A} receptor positive allosteric modulator (PAM) in an amount sufficient to promote the replication, and/or survival, and/or growth, and/or mass, and/or function of beta cells in said mammal.

Embodiment 2: A method of embodiment 1, wherein said GABA_{A} receptor positive allosteric modulator (PAM) is administered in conjunction with a GABA receptor activating ligand.

Embodiment 3: The method of embodiment 2, wherein the GABA receptor activating ligand when used in conjunction with said PAM is more effective to promote the replication, and/or survival, and/or function of beta cells in said mammal than when either agent is administered alone.

Embodiment 4: The method of embodiment 3, wherein the GABA receptor activating ligand when used in conjunction with a PAM is at least 10%, or at least 20%, or at least 30%, or at least 40%, or at least 50%, or at least 60%, or at least 70%, or at least 80%, or at least 90%, or at least 100%, or at least 1.2 fold, or at least 1.5 fold, or at least 2 fold, or at least 3 fold, or at least 4 fold, or at least 5 fold, or at least 10 fold is more effective to promote the replication, and/or survival, and/or function of beta cells in said mammal than when either agent is administered alone.

Embodiment 5: The method of embodiment 2, wherein the GABA receptor activating ligand is used at a lower dosage than would be used to achieve the same effect on beta cell replication, and/or survival, and/or function when used alone and/or said positive allosteric modulator is used at a lower dosage than would be used to achieve the activity for which the PAM is designed and/or approved if the PAM were used alone.

Embodiment 6: The method of embodiment 5, wherein the GABA receptor activating ligand used at less than about 95%, or less than about 90%, or less than about 80%, or less than about 70%, or less than about 60%, or less than about 50%, or less than about 40%, or less than about 30%, or less than about 20%, or less than about 10% of the dosage that would be used to achieve the same effect on beta cell replication, and/or survival, and/or function when the GABA receptor activating ligand is used alone.

Embodiment 7: The method according to any one of embodiments 5-6, wherein said positive allosteric modulator is used at less than about 95%, or less than about 90%, or less than about 80%, or less than about 70%, or less than about 60%, or less than about 50%, or less than about 40%, or less than about 30%, or less than about 20%, or less than about 10% of the dosage that that would be used to achieve the activity for which the PAM is designed and/or approved if the PAM were used alone.

Embodiment 8: The method according to any one of embodiments 5-7, where the GABA receptor activating ligand is used at a lower dosage than would be used to achieve the same effect on beta cell replication, and/or survival, and/or function when used alone and/or said positive allosteric modulator is used at a subtherapeutic dosage.

Embodiment 9: The method according to any one of embodiments 1-8, wherein said administration promotes replication of beta cells.

Embodiment 10: The method according to any one of embodiments 1-9, wherein said administration increases mass of beta cells.

Embodiment 11: The method according to any one of embodiments 1-10, wherein said administration promotes survival of beta cells.

Embodiment 12: The method according to any one of embodiments 1-11, wherein said administration promotes function of beta cells.

Embodiment 13: The method of embodiment 12, wherein said administration increases the insulin content and/or the amount of insulin secreted by beta cells.

Embodiment 14: The method of embodiment 12, wherein said administration promotes function by increasing the number of insulin positive beta cells.

Embodiment 15: The method according to any one of embodiments 1-14, wherein said mammal is a human.

Embodiment 16: The method of embodiment 15, wherein said mammal is a human diagnosed with type I diabetes.

Embodiment 17: The method of embodiment 15, wherein said mammal is diagnosed as pre-diabetic.

Embodiment 18: The method according to any one of embodiments 1-14, wherein said mammal is a non-human mammal.

Embodiment 19: The method according to any one of embodiments 1-18, wherein said GABA receptor activating ligand and said PAM act synergistically to improve the replication, and/or survival, and/or growth, and/or function of said beta cells.

Embodiment 20: The method according to any one of embodiments 1-19, wherein said PAM comprises an agent selected from the group consisting of AP325, and AP3.

Embodiment 21: The method of embodiment 20, wherein said PAM comprises AP325.

Embodiment 22: The method of embodiment 21, wherein said AP325 is administered at a dosage lower than that used for neuropathic pain or spinal cord injury.

Embodiment 23: The method according to any one of embodiments 1-19, wherein said PAM comprises an agent selected from the group consisting of a barbituate, a benzodiazepine, a quinazolinone, and a neurosteroid.

Embodiment 24: The method of embodiment 23, wherein said PAM comprises a barbituate.

Embodiment 25: The method of embodiment 24, wherein said PAM comprises a barbiturate selected from the group consisting of allobarbital (5,5-diallylbarbiturate), amobarbital (5-ethyl-5-isopentyl-barbiturate), aprobarbital (5-allyl-5-isopropyl-barbiturate), alphenal (5-allyl-5-phenyl-barbiturate), barbital (5,5-diethylbarbiturate), brallobarbital (5-allyl-5-(2-bromo-allyl)-barbiturate), pentobarbital (5-ethyl-5-(1-methylbutyl)-barbiturate), phenobarbital (5-ethyl-5-phenylbarbiturate), secobarbital (5-[(2R)-pentan-2-yl]-5-prop-2-enyl-barbiturate).

Embodiment 26: The method of embodiment 23, wherein said PAM comprises a benzodiazepine.

Embodiment 27: The method of embodiment 26, wherein said PAM comprises a benzodiazepine selected from the group consisting of alprazolam, bromazepam, chlordiazepoxide, midazolam, clonazepam, clorazepate, diazepam, estazolam, flurazepam, halazepam, ketazolam, lorazepam, nitrazepam, oxazepam, prazepam, quazepam, temazepam, and triazolam.

Embodiment 28: The method of embodiment 26, wherein said PAM comprises alprazolam.

Embodiment 29: The method of embodiment 28, wherein said alprazolam is administered at a dosage less than that used to treat used to treat an anxiety disorder, a panic disorder, and/or anxiety caused by depression.

Embodiment 30: The method of embodiment 28, wherein said alprazolam is administered as an immediate release tablet less than 1.5 mg orally per day or less than 1.0 mg orally per day, or less than 0.5 mg orally per day, or as an extended release tablet less than 0.5 mg orally per day, or less than about 0.4 mg orally per day or less than about 3 mg orally per day.

Embodiment 31: The method of embodiment 26, wherein said PAM comprises midazolam.

Embodiment 32: The method of embodiment 31, wherein said midazolam is administered at a dosage less than that used to reduce anxiety, or producing drowsiness or anesthesia before medical procedures or surgery, or to maintain sedation or anesthesia.

Embodiment 33: The method of embodiment 31, wherein said midazolam is administered at a dosage less than 1 mg IV, or less than about 0.8 mg IV, or less than about 0.5 mg IV, or less than about 0.01 mg/kg IV, or less than about 0.07 mg/kg IM, or less than about 0.05 mg/kg IM, or less than about 0.03 mg/kg IM, or less than about 0.01 mg/kg.

Embodiment 34: The method of embodiment 26, wherein said PAM comprises clonazepam.

Embodiment 35: The method of embodiment 34, wherein said clonazepam is administered at a dosage less than that used to treat seizure disorders (including absence seizures or Lennox-Gastaut syndrome), or less than that used to treat panic disorder (including agoraphobia) in adults.

Embodiment 36: The method of embodiment 34, wherein said clonazepam is administered at a dosage less than about 0.5 mg orally per day, or less than about 0.25 mg orally per day, or less than bout 0.01 mg/kg/day, or less than about 0.005 mg/kg/day.

Embodiment 37: The method of embodiment 23, wherein said PAM comprises a neurosteroid.

Embodiment 38: The method of embodiment 37, wherein said PAM comprises a neurosteroid selected from the group consisting of allopregnanolone (3α-hydroxy-5α-pregnan-20-one), and pregnanolone.

Embodiment 39: The method according to any one of embodiments 2-38, wherein said GABA receptor activating ligand comprises GABA.

Embodiment 40: The method according to any one of embodiments 2-38, wherein said GABA receptor activating ligand comprises an agent selected from the group consisting of bamaluzole, gabamide, GABOB, gaboxadol, ibotenic acid, isoguvacine, isonipecotic acid, muscimol, phenibut, picamilon, progabide, quisqualamine, progabide acid (SL 75102), thiomuscimol, pregabalin, vigabatrin, 6-aminonicotinic acid, homotaurine, and XP13512 [(±)-1-([(α-isobutanoyloxyethoxy) carbonyl] aminomethyl)-1-cyclohexane acetic acid].

Embodiment 41: The method according to any one of embodiments 1-40, wherein said method increases beta cell survival after islet transplantation.

Embodiment 42: The method according to any one of embodiments 1-41, wherein said method increases beta cell replication in islets after islet transplantation.

Embodiment 43: The method according to any one of embodiments 1-40, wherein said method is performed after islet implantation in order to reduce β-cell loss due to hypoxia and stress.

Embodiment 44: The method according to any one of embodiments 1-43, where said method is performed for up to 3 days post implantation, or for up to 1 week post implantation, or for up to 2 weeks post implantation, or for up to 3 weeks post implantation, or for up to 4 weeks post implantation, or for up to 5 weeks post implantation, or for up to 6 weeks post implantation, or for up to 7 weeks post implantation, or for up to 8 weeks post implantation, or for up to 3 months post implantation, or for up to 4 months post implantation, or for up to 5 months post implantation, or for up to 6 months post implantation.

Embodiment 45: The method according to any one of embodiments 2-44, wherein said GABA receptor activating ligand is not an alcohol.

Embodiment 46: The method according to any one of embodiments 2-45, wherein said GABA receptor activating ligand is not a kavalactone.

Embodiment 47: The method according to any one of embodiments 2-46, wherein said GABA receptor activating ligand is not skullcap or a skullcap constituent.

Embodiment 48: The method according to any one of embodiments 2-47, wherein said GABA receptor activating ligand is not valerian or a valerian constituent.

Embodiment 49: The method according to any one of embodiments 2-48, wherein said GABA receptor activating ligand is not a volatile gas.

Embodiment 50: The method according to any one of embodiments 1-49, wherein said mammal is not under treatment for one or more conditions selected from the group consisting of neuropathic pain, spinal cord injury, an anxiety disorder, a panic disorder, anxiety caused by depression, a seizure disorder (including absence seizures or Lennox-Gastaut syndrome), and catamenial epilepsy.

Embodiment 51: The method according to any one of embodiments 1-50, wherein said PAM is not administered to produce drowsiness or anesthesia before a medical procedure or surgery, or to maintain sedation or anesthesia.

Embodiment 52: The method according to any one of embodiments 1-51, wherein said PAMS that are BBB-permeable are administered at doses below those used for CNS indications.

Embodiment 53: A pharmaceutical formulation comprising a GABA receptor activating ligand and a GABA_{A} receptor positive allosteric modulator (PAM).

Embodiment 54: The formulation of embodiment 53, wherein the GABA receptor activating ligand is present at a lower unit dosage than in a therapeutic formulation comprising a GABA receptor activating ligand alone and/or the PAM is at a lower unit dosage than in a therapeutic formulation comprising the PAM alone.

Embodiment 55: The formulation of embodiment 54, wherein the GABA receptor activating ligand is present at a lower unit dosage than in a therapeutic formulation comprising GABA or a GABA analog alone.

Embodiment 56: The formulation of embodiment 55, wherein the GABA receptor activating ligand present at less than about 95%, or less than about 90%, or less than about 80%, or less than about 70%, or less than about 60%, or less than about 50%, or less than about 40%, or less than about 30%, or less than about 20%, or less than about 10% of the dosage that would be provided to achieve the same effect on beta cell replication, and/or survival, and/or function when the GABA receptor activating ligand is present alone.

Embodiment 57: The formulation according to any one of embodiments of embodiment 53-56, wherein the PAM is at a lower unit dosage than in a therapeutic formulation comprising the PAM alone.

Embodiment 58: The formulation of embodiment 57, wherein said positive allosteric modulator is present at less than about 95%, or less than about 90%, or less than about 80%, or less than about 70%, or less than about 60%, or less than about 50%, or less than about 40%, or less than about 30%, or less than about 20%, or less than about 10% of the amount that that would be present to achieve the activity for which the PAM is designed and/or approved if the PAM were used alone.

Embodiment 59: The formulation according to any one of embodiments 53-58, wherein said GABA or GABA analog and said PAM are present in a concentration sufficient to provide synergistic activity in stimulating replication of cells expressing a GABA_{A} receptor.

Embodiment 60: The formulation according to any one of embodiments 53-58, wherein said GABA receptor activating ligand and said PAM are present in a concentration sufficient to provide synergistic activity in stimulating replication of beta cells and/or promoting survival of beta cells and/or improving function of beta cells.

Embodiment 61: The formulation according to any one of embodiments 53-60, wherein said GABA receptor activating ligand and said PAM are present in a concentration sufficient provide at least 10%, or at least 20%, or at least 30%, or at least 40%, or at least 50%, or at least 60%, or at least 70%, or at least 80%, or at least 90%, or at least 100%, or at least 1.2 fold, or at least 1.5 fold, or at least 2 fold, or at least 3 fold, or at least 4 fold, or at least 5 fold, or at least 10 fold greater efficacy in promoting the replication, and/or survival, and/or function of beta cells in a mammal than when either agent is administered alone.

Embodiment 62: The formulation according to any one of embodiments 53-61, wherein said PAM comprises an agent selected from the group consisting of a barbituate, a benzodiazepine, a quinazolinone, and a neurosteroid.

Embodiment 63: The formulation of embodiment 62, wherein said PAM comprises a barbituate.

Embodiment 64: The formulation of embodiment 63, wherein said PAM comprises a barbiturate selected from the group consisting of allobarbital (5,5-diallylbarbiturate), amobarbital (5-ethyl-5-isopentyl-barbiturate), aprobarbital (5-allyl-5-isopropyl-barbiturate), alphenal (5-allyl-5-phenyl-barbiturate), barbital (5,5-diethylbarbiturate), brallobarbital (5-allyl-5-(2-bromo-allyl)-barbiturate), pentobarbital (5-ethyl-5-(1-methylbutyl)-barbiturate), phenobarbital (5-ethyl-5-phenylbarbiturate), secobarbital (5-[(2R)-pentan-2-yl]-5-prop-2-enyl-barbiturate).

Embodiment 65: The formulation of embodiment 62, wherein said PAM comprises a benzodiazepine.

Embodiment 66: The formulation of embodiment 65, wherein said PAM comprises a benzodiazepine selected from the group consisting of alprazolam, bromazepam, chlordiazepoxide, midazolam, clonazepam, clorazepate, diazepam, estazolam, flurazepam, halazepam, ketazolam, lorazepam, nitrazepam, oxazepam, prazepam, quazepam, temazepam, and triazolam.

Embodiment 67: The formulation of embodiment 65, wherein said PAM comprises alprazolam.

Embodiment 68: The formulation of embodiment 65, wherein said PAM comprises midazolam.

Embodiment 69: The formulation of embodiment 65, wherein said PAM comprises clonazepam.

Embodiment 70: The formulation of embodiment 62, wherein said PAM comprises a neurosteroid.

Embodiment 71: The formulation of embodiment 70, wherein said PAM comprises a neurosteroid selected from the group consisting of allopregnanolone, and pregnanolone.

Embodiment 72: The formulation according to any one of embodiments 53-71, wherein said GABA receptor activating ligand comprises GABA.

Embodiment 73: The formulation according to any one of embodiments 53-71, wherein said GABA receptor activating ligand an agent selected from the group consisting of bamaluzole, gabamide, GABOB, gaboxadol, ibotenic acid, isoguvacine, isonipecotic acid, muscimol, phenibut, picamilon, progabide, quisqualamine, progabide acid (SL 75102), thiomuscimol, pregabalin, vigabatrin, 6-aminonicotinic acid, homotaurine, and XP13512 [(±)-1-([(α-isobutanoyloxyethoxy) carbonyl] aminomethyl)-1-cyclohexane acetic acid].

Embodiment 74: The formulation according to any one of embodiments 53-73, wherein said GABA receptor activating ligand is not an alcohol.

Embodiment 75: The formulation according to any one of embodiments 53-74, wherein said GABA receptor activating ligand is not a kavalactone.

Embodiment 76: The formulation according to any one of embodiments 53-75, wherein said GABA receptor activating ligand is not skullcap or a skullcap constituent.

Embodiment 77: The formulation according to any one of embodiments 53-76, wherein said GABA receptor activating ligand is not valerian or a valerian constituent.

Embodiment 78: A kit for promoting the replication in a mammal of cells that express GABA_{A} receptors, said kit comprising: a container containing a GABA receptor activating ligand; and a container containing a GABA_{A} receptor positive allosteric modulator (PAM).

Embodiment 79: The kit of embodiment 78 where the GABA receptor activating ligand and the PAM are in the same container.

Embodiment 80: The kit of embodiment 78 where the GABA receptor activating ligand and the PAM are in separate containers.

Embodiment 81: The kit according to any one of embodiments 78-80, wherein the GABA receptor activating ligand is present at a lower unit dosage than in a therapeutic formulation comprising GABA or a GABA analog alone and/or the PAM is at a lower unit dosage than in a therapeutic formulation comprising the PAM alone.

Embodiment 82: The kit according to any one of embodiments 78-80, wherein the GABA receptor activating ligand is present at a lower unit dosage than in a therapeutic formulation comprising GABA or a GABA analog alone.

Embodiment 83: The kit of embodiment 82, wherein the GABA receptor activating ligand present at less than about 95%, or less than about 90%, or less than about 80%, or less than about 70%, or less than about 60%, or less than about 50%, or less than about 40%, or less than about 30%, or less than about 20%, or less than about 10% of the dosage that would be provided to achieve the same effect on beta cell replication, and/or survival, and/or function when the GABA receptor activating ligand is present alone.

Embodiment 84: The kit according to any one of embodiments 78-83, wherein the PAM is at a lower unit dosage than in a therapeutic formulation comprising the PAM alone.

Embodiment 85: The kit according to any one of embodiments 78-84 wherein said positive allosteric modulator is present at less than about 95%, or less than about 90%, or less than about 80%, or less than about 70%, or less than about 60%, or less than about 50%, or less than about 40%, or less than about 30%, or less than about 20%, or less than about 10% of the amount that that would be present to achieve the activity for which the PAM is designed and/or approved if the PAM were used alone.

Embodiment 86: The kit according to any one of embodiments 78-85, wherein said GABA receptor activating ligand and said PAM are present in a concentration sufficient to provide synergistic activity in stimulating replication of cells expressing a GABA_{A} receptor.

Embodiment 87: The kit of according to any one of embodiments 78-85, wherein said GABA receptor activating ligand and said PAM are present in a concentration sufficient to provide synergistic activity in stimulating replication of beta cells and/or promoting survival of beta cells and/or improving function of beta cells.

Embodiment 88: The kit according to any one of embodiments 78-87, wherein said GABA receptor activating ligand and said PAM are present in a concentration sufficient provide at least 10%, or at least 20%, or at least 30%, or at least 40%, or at least 50%, or at least 60%, or at least 70%, or at least 80%, or at least 90%, or at least 100%, or at least 1.2 fold, or at least 1.5 fold, or at least 2 fold, or at least 3 fold, or at least 4 fold, or at least 5 fold, or at least 10 fold greater efficacy in promoting the replication, and/or survival, and/or function of beta cells in a mammal than when either agent is administered alone.

Embodiment 89: The kit according to any one of embodiments 78-88, wherein said PAM comprises an agent selected from the group consisting of a barbituate, a benzodiazepine, a quinazolinone, and a neurosteroid.

Embodiment 90: The kit of embodiment 89, wherein said PAM comprises a barbituate.

Embodiment 91: The kit of embodiment 90, wherein said PAM comprises a barbiturate selected from the group consisting of allobarbital (5,5-diallylbarbiturate), amobarbital (5-ethyl-5-isopentyl-barbiturate), aprobarbital (5-allyl-5-isopropyl-barbiturate), alphenal (5-allyl-5-phenyl-barbiturate), barbital (5,5-diethylbarbiturate), brallobarbital (5-allyl-5-(2-bromo-allyl)-barbiturate), pentobarbital (5-ethyl-5-(1-methylbutyl)-barbiturate), phenobarbital (5-ethyl-5-phenylbarbiturate), secobarbital (5-[(2R)-pentan-2-yl]-5-prop-2-enyl-barbiturate).

Embodiment 92: The kit of embodiment 89, wherein said PAM comprises a benzodiazepine.

Embodiment 93: The kit of embodiment 92, wherein said PAM comprises a benzodiazepine selected from the group consisting of alprazolam, bromazepam, chlordiazepoxide, midazolam, clonazepam, clorazepate, diazepam, estazolam, flurazepam, halazepam, ketazolam, lorazepam, nitrazepam, oxazepam, prazepam, quazepam, temazepam, and triazolam.

Embodiment 94: The kit of embodiment 92, wherein said PAM comprises alprazolam.

Embodiment 95: The kit of embodiment 92, wherein said PAM comprises clonazepam.

Embodiment 96: The kit of embodiment 92, wherein said PAM comprises midazolam.

Embodiment 97: The kit of embodiment 89, wherein said PAM comprises a neurosteroid.

Embodiment 98: The kit of embodiment 97, wherein said PAM comprises a neurosteroid selected from the group consisting of allopregnanolone, and pregnanolone.

Embodiment 99: The kit according to any one of embodiments 78-98, wherein said GABA receptor activating ligand comprises GABA.

Embodiment 100: The kit according to any one of embodiments 78-98, wherein said GABA receptor activating ligand comprises an agent selected from the group consisting of bamaluzole, gabamide, GABOB, gaboxadol, ibotenic acid, isoguvacine, isonipecotic acid, muscimol, phenibut, picamilon, progabide, quisqualamine, progabide acid (SL 75102), thiomuscimol, pregabalin, vigabatrin, 6-aminonicotinic acid, homotaurine, and XP13512 [(±)-1-([(α-isobutanoyloxyethoxy) carbonyl] aminomethyl)-1-cyclohexane acetic acid].

Embodiment 101: The kit according to any one of embodiments 78-100, wherein said GABA receptor activating ligand is not an alcohol.

Embodiment 102: The kit according to any one of embodiments 78-101, wherein said GABA receptor activating ligand is not a kavalactone.

Embodiment 103: The kit according to any one of embodiments 78-102, wherein said GABA receptor activating ligand is not skullcap or a skullcap constituent.

Embodiment 104: The kit according to any one of embodiments 78-103, wherein said GABA receptor activating ligand is not valerian or a valerian constituent.

### Definitions

The term “GABA receptor activating ligand” refers to an agent that is an agonist for one or more of the GABA receptors. In certain embodiments, the GABA receptor activating ligand is an agonist for at least the GABA_{A} receptor.

The terms “positive allosteric modulator of the GABA_{A} receptor” or (PAM) refers to a molecule that increase the activity of the GABA_{A} receptor protein in the vertebrate central nervous system. Unlike GABA_{A} receptor agonists, GABA_{A} PAMs do not bind at the same active site as the gamma-aminobutyric acid (GABA) neurotransmitter molecule. In various embodiments, the PAMs trigger or potentiate the GABA_{A} receptor to open its chloride channel.

The term “beta cell function” refers to the function of beta cells, particularly with respect to insulin production, secretion, and β-cell area. Increase in beta cell function refers to an increase in insulin content of beta cells and/or an increase in insulin secretion by beta cells and/or an increase in the number of insulin positive beta cells.

The term “subtherapeutic dosage” when used with respect to a PAM refers to a dosage below the approved and/or recommended and/or recognized dosage of the PAM when used for the activity for which the PAM was originally designed and/or approved. Thus, for example, the subtherapeutic dosage of a benzodiazepine such as alprazolam refers to a dosage below the approved and/or recommended and/or recognized dosage of that benzodiazepine for depression, panic disorder, and/or anxiety. In certain embodiments the subtherapeutic dosage is less than 90% or less than about 80%, or less than about 70%, or less than about 60%, or less than about 50%, or less than about 40%, or less than about 30%, or less than about 20%, or less than about 10% of the recommended dosage for the activity for which the PAM was designed and/or approved (e.g., depression, panic disorder, and/or anxiety).

The phrases “in conjunction with” or “in combination with” when used in reference to the use of the active agent(s) described herein (e.g., one or more GABA receptor activating ligand(s)) in conjunction with one or more other drugs described herein (e.g., one or more PAMs) indicates that the GABA receptor activating ligand(s) and the PAM(s) are administered so that there is at least some chronological overlap in their physiological activity on the organism, and in particular on beta cells. Thus, in various embodiments, the GABA receptor activating ligand (s) and PAM(s) can be administered simultaneously and/or sequentially. In sequential administration there may even be some substantial delay (e.g., minutes or even hours or days) before administration of the second moiety as long as the first administered drug/agent has exerted some physiological alteration on the organism when the second administered agent is administered or becomes active in the organism.

### BRIEF DESCRIPTION OF THE DRAWINGS

FIG. 1 shows a schematic diagram illustrating GABA receptor subunits. This fig only shows the benzodiazepine binding site. There are other binding sites for other classes of PAMs.

FIG. 2. Human islets (50/well) were incubated in media with, or without, alprazolam (30 ng/ml) for 4 days in the presence of ³H thymidine. Data shown are the average rate of proliferation relative to that of cultures with media alone (designated as 1). N=two independent studies with triplicate cultures. Data was very similar in both studies.

FIG. 3 Human islets (50 IEQ/well) were incubated with the indicated dosage of GABA, together with, or without, alprazolam (30 ng/ml) for 4 days in the presence of ₃H thymidine. Data shown are the average rate of proliferation relative to that of cultures without GABA or alprazolam (designated as 1). N=two independent studies

FIG. 4, panels A and B, shows that with midazolam enhanced the proliferation of INS-1 cells. Panel A) Shows the results over a broad dosage range in steps of 10-fold. Panel B) Shows results over a smaller dose range in smaller dose increments.

FIG. 5, panels An and B, shows that treatment with midazolam or clonazepam enhances GABA-induced INS-1 cell proliferation in vitro.

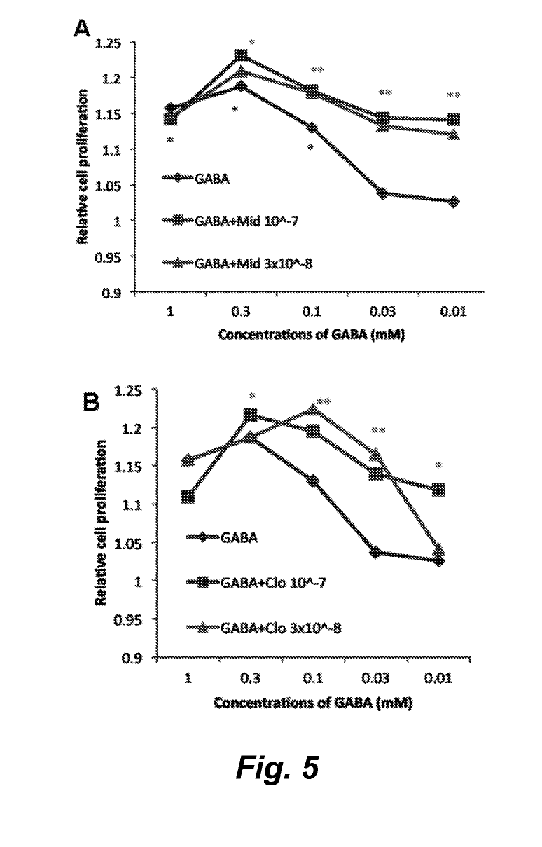

FIG. 6 shows that treatment with AP325 or AP3 does not stimulate INS-1 cell proliferation in vitro. Data are expressed as the mean±SD of % cell proliferation, relative to the control without any treatment from at least three separate experiments. The control cell proliferation was designated as 100.

FIG. 7 shows that AP325 enhances GABA-stimulated INS-1 cell proliferation in vitro. Data are expressed as the mean±SD of cell proliferation relative to the control without any treatment from at least three separate experiments. The control cell proliferation was designated as 1.

FIG. 8 shows that AP3 enhances GABA-induced INS-1 cell proliferation in vitro. Data are expressed as the mean±SD of relative cell proliferation to the control without any treatment from at three separate experiments. The control cell proliferation was designated as 1. Treatments that induced significant differences from the control are stated in the Example 3).

FIG. 9, panels A-H: Panel A) GAD enzymatic activity in INS-1 cells. INS-1 cells and 293T cells (harvested in growth phase), as well as fresh mouse brain and human islets were stored at −80 C until use. The samples were homogenized into GAD activity assay buffer with protease inhibitors. The GAD enzymatic activity within homogenates was assessed using a standard CO₂ trapping assay (in quadruplicate) as previously described (Kaufman et al. (1991) J. Neurochem. 56: 720-723). Data shown is average CPM+/−SEM from a representative assay from three experiments. Panel B) Effect of PAMs on INS-1 cell proliferation. INS-1 cells were cultured with the indicated PAM at a dose range of 10⁻⁹ to 10⁻⁶ M and assessed for their proliferation. Data shown are the average rate of proliferation relative to that of cultures with media alone (designated as 1). Alprazolam (panel C), midazolam (panel D), clonazepam (panel E), AP325 (panel F), or AP3 (panel G), were incubated at the indicated concentrations with a dose range of GABA. Control cultures were incubated with GABA alone at the indicated concentration. ^{††††}p<0.001 versus control cultures with media alone. *p<0.05, **p<0.01, ***p<0.001 versus cultures with the same dose of GABA. H) INS-1 cells were cultured with GABA (0.3 mM) and midazolam or clonazepam (100 nM) with, or without, the TSPO inhibitor PK11195 (1 uM) for 48 hrs.

FIG. 10 shows that alprazolam enhances human islet cell replication. Fresh human islets were treated with the indicated dosage of GABA, together with, or without, the indicated PAM as described in Methods. Data shown are the average rate of proliferation relative to that of cultures with media alone (designated as 1). N=two independent studies with triplicate cultures. The results were very similar in both studies. *p<0.05

FIG. 11 shows that the GABA-binding site blocker/inhibit bicuculline ablates the ability of alprazolam to enhance islet cell replication. Human islets were incubated with alprazolam (100 nM) together with a standard dose range of bicuculline (0-50 uM). Data shown are the average rate of proliferation relative to that of cultures with media alone (designated as 1).

FIG. 12 shows that alprazolam enhances GABA's ability to promote human islet cell replication. Human islets were incubated with a dose range of GABA together with alprazolam (100 ng/ml) for 4 days with ³H thymidine. Data shown are the average rate of proliferation relative to that of cultures with media alone (designated as 1) in a representative study. N=two independent studies with triplicate cultures. ^{††}p<0.01 and ^{††\p<}0.001 for GABA, or GABA+alprazolam vs. control media. *p<0.05 and ***P<0.01 for GABA+alprazolam vs. GABA alone.

### DETAILED DESCRIPTION

The compositions and methods described herein pertain to the discovery that GABA receptor activating ligands in combination with a positive allosteric modulator of GABA_{A} receptors can promote the replication, and/or survival, and/or function of beta cells in mammal. Moreover use of the GABA receptor activating ligand in combination with the positive allosteric modulator provides a significantly greater effect on beta cell replication and/or survival, and/or function than the GABA receptor activating ligand used alone. Without being bound to a particular theory it appears that the combination of a GABA receptor activating ligand and a PAM has a synergistic effect on beta cell replication and/or survival, and/or function.

In view of the improved effect offered by the combination of the GABA receptor activating ligand and the PAM, it will be recognized that either the GABA receptor activating ligand and/or the PAM can be used at a dosage lower than would be used if the GABA receptor activating ligand or PAM was used alone to promote the replication, and/or survival, and/or function of beta cells in mammal (e.g., in a mammal receiving an islet cell transplant).

Accordingly, in various embodiments, methods of promoting the replication, and/or survival, and/or function of beta cells in mammal are provided where the methods involve administering to a mammal in need thereof (e.g., a subject receiving an islet cell transplant) a GABA receptor activating ligand in conjunction with a GABA_{A} receptor positive allosteric modulator (PAM) where the GABA receptor activating ligand is used at a lower dosage than would be used to achieve the same effect on beta cell replication, and/or survival, and/or function than when used alone and/or said positive allosteric modulator is used at a subtherapeutic dosage. In certain embodiments the administration of both agents promotes replication of beta cells. In certain embodiments the administration of both agents promotes survival of beta cells. In certain embodiments the administration of both agents promotes function of beta cells (e.g. increases insulin content and/or secretion). In certain embodiments the administration of both agents increases the number of insulin positive beta cells.

In certain embodiments pharmaceutical formulations are provided that comprise a GABA receptor activating ligand and a GABA_{A} receptor positive allosteric modulator (PAM). In certain embodiments kits for the practice of the methods described herein are also contemplated. In various embodiments such kits comprises a container containing a GABA receptor activating ligand and a container containing a GABA_{A} receptor positive allosteric modulator (PAM), and optionally instructional materials teaching, inter alia, the use of these agents in the methods described herein.

### GABA Receptor Activating Ligands

GABA receptor activating ligands are well known to those of skill in the art. Such ligands include for example gamma-aminobutyric acid (GABA) the native ligand for the GABA receptor. Other GABA receptor activating ligands include, but are not limited to homotaurine, bamaluzole, gabamide, GABOB, gaboxadol, ibotenic acid, isoguvacine, isonipecotic acid, muscimol, phenibut, picamilon, progabide, quisqualamine, progabide acid (SL 75102), thiomuscimol, pregabalin, vigabatrin, 6-aminonicotinic acid, XP13512 ((±)-1-([(α-isobutanoyloxyethoxy) carbonyl] aminomethyl)-1-cyclohexane acetic acid), and the like.

These GABA receptor activating ligands are intended to be illustrative and non-limiting. Other GABA receptor activating ligands are known to those of skill in the art and their use in the methods, formulations, and kits described herein is contemplated.

### GABA_{A} Receptor Positive Allosteric Modulator (PAM)

Positive allosteric modulators (PAMs) of GABAA are well known to those of skill in the art. Illustrative PAMS include, but are not limited to alcohols (e.g., ethanol, isopropanol), avermectins (e.g., ivermectin), barbiturates (e.g., phenobarbital), benzodiazepines, bromides (e.g., potassium bromide, carbamates (e.g., meprobamate, carisoprodol), chloralose, chlormezanone, clomethiazole, dihydroergolines (e.g., ergoloid (dihydroergotoxine)), etazepine, etifoxine, imidazoles (e.g., etomidate), kavalactones (found in kava), loreclezole, neuroactive steroids (e.g., allopregnanolone, ganaxolone), nonbenzodiazepines (e.g., zaleplon, zolpidem, zopiclone, eszopiclone), petrichloral, phenols (e.g., propofol), piperidinediones (e.g., glutethimide, methyprylon), propanidid, pyrazolopyridines (e.g., etazolate), quinazolinones (e.g., methaqualone), skullcap constituents (e.g. constituents of Scutellaria sp. including, but not limited to flavonoids such as baicalein), stiripentol, sulfonylalkanes (e.g., sulfonmethane, tetronal, trional), valerian constituents (e.g., valeric acid, valerenic acid), and certain volatiles/gases (e.g., chloral hydrate, chloroform, diethyl ether, sevoflurane). In various embodiments the PAMs used in combination with the GABA receptor activating ligands exclude alcohols, and/or kavalactones, and/or skullcap or skullcap constituents, and/or valerian or valerian constituents, and/or volatile gases.

In certain embodiments the PAM comprises an agent selected from the group consisting of a barbituate, a benzodiazepine, a quinazolinone, and a neurosteroid. Illustrative barbituates include, but are not limited to allobarbital (5,5-diallylbarbiturate), amobarbital (5-ethyl-5-isopentyl-barbiturate), aprobarbital (5-allyl-5-isopropyl-barbiturate), alphenal (5-allyl-5-phenyl-barbiturate), barbital (5,5-diethylbarbiturate), brallobarbital (5-allyl-5-(2-bromo-allyl)-barbiturate), pentobarbital (5-ethyl-5-(1-methylbutyl)-barbiturate), phenobarbital (5-ethyl-5-phenylbarbiturate), secobarbital (5-[(2R)-pentan-2-yl]-5-prop-2-enyl-barbiturate), and the like.

Illustrative benzodiazepines include, but are not limited to alprazolam, bromazepam, chlordiazepoxide, clonazepam, clorazepate, diazepam, estazolam, flurazepam, halazepam, ketazolam, lorazepam, nitrazepam, oxazepam, prazepam, quazepam, temazepam, triazolam, and the like.

Illustrative neurosteroids include, but are not limited to allopregnanolone, and pregnanolone.

In certain embodiments the PAM of particular use comprises alprazolam (XANAX®).

These PAMs are intended to be illustrative and non-limiting. Other PAMs are known to those of skill in the art and their use in the methods, formulations, and kits described herein is contemplated.

### Combined Formulations.

In various embodiments pharmaceutical formulations are contemplated that comprise one or more GABA receptor activating ligands and one or more positive allosteric modulators of the GABA_{A} receptor. In certain embodiments the GABA receptor activating ligand is present at a lower unit dosage than in a therapeutic formulation comprising a GABA receptor activating ligand alone and/or the PAM is at a lower unit dosage than in a typical, and/or approved, and/or recommended therapeutic formulation comprising the PAM alone.

In certain embodiments GABA receptor ligand in the combined formulation comprises one or more of gamma-aminobutyric acid (GABA), homotaurine, bamaluzole, gabamide, GABOB, gaboxadol, ibotenic acid, isoguvacine, isonipecotic acid, muscimol, phenibut, picamilon, progabide, quisqualamine, progabide acid (SL 75102), thiomuscimol, pregabalin, vigabatrin, 6-aminonicotinic acid, XP13512 ((±)-1-([(α-isobutanoyloxyethoxy) carbonyl] aminomethyl)-1-cyclohexane acetic acid), and the like, e.g., as described herein.

In certain embodiments the PAM in the combined formulation comprises one or more of an alcohol (e.g., ethanol, isopropanol), avermectins (e.g., ivermectin), barbiturates (e.g., phenobarbital), benzodiazepines, bromides (e.g., potassium bromide, carbamates (e.g., meprobamate, carisoprodol), chloralose, chlormezanone, clomethiazole, dihydroergolines (e.g., ergoloid (dihydroergotoxine)), etazepine, etifoxine, imidazoles (e.g., etomidate), kavalactones (found in kava), loreclezole, neuroactive steroids (e.g., allopregnanolone, ganaxolone), nonbenzodiazepines (e.g., zaleplon, zolpidem, zopiclone, eszopiclone), petrichloral, phenols (e.g., propofol), piperidinediones (e.g., glutethimide, methyprylon), propanidid, pyrazolopyridines (e.g., etazolate), quinazolinones (e.g., methaqualone), skullcap constituents (e.g. constituents of Scutellaria sp. including, but not limited to flavonoids such as baicalein), stiripentol, sulfonylalkanes (e.g., sulfonmethane, tetronal, trional), valerian constituents (e.g., valeric acid, valerenic acid). In various embodiments the PAMs used in combination with the GABA receptor activating ligands exclude alcohols, and/or kavalactones, skullcap or skullcap constituents, and the like, e.g., as described herein.

In certain embodiments the combined formulation comprises GABA and alprazolam.

The active agent(s) (e.g., GABA receptor activating ligand(s) and PAM(s) described herein) can be formulated and administered in the “native” form or, if desired, in the form of salts, esters, amides, prodrugs, derivatives, and the like, provided the salt, ester, amide, prodrug or derivative is suitable pharmacologically, i.e., effective in the present method(s). Salts, esters, amides, prodrugs and other derivatives of the active agents can be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience, and as described above.

For example, a pharmaceutically acceptable salt can be prepared for any of the agent(s) described herein (e.g., GABA receptor activating ligand(s) and PAM(s) described herein) having a functionality capable of forming a salt. A pharmaceutically acceptable salt is any salt that retains the activity of the parent compound and does not impart any deleterious or untoward effect on the subject to which it is administered and in the context in which it is administered.

In various embodiments pharmaceutically acceptable salts may be derived from organic or inorganic bases. The salt may be a mono or polyvalent ion. Of particular interest are the inorganic ions, lithium, sodium, potassium, calcium, and magnesium. Organic salts may be made with amines, particularly ammonium salts such as mono-, di- and trialkyl amines or ethanol amines. Salts may also be formed with caffeine, tromethamine and similar molecules.

Methods of formulating pharmaceutically active agents as salts, esters, amide, prodrugs, and the like are well known to those of skill in the art. For example, salts can be prepared from the free base using conventional methodology that typically involves reaction with a suitable acid. Generally, the base form of the drug is dissolved in a polar organic solvent such as methanol or ethanol and the acid is added thereto. The resulting salt either precipitates or can be brought out of solution by addition of a less polar solvent. Suitable acids for preparing acid addition salts include, but are not limited to both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt can be reconverted to the free base by treatment with a suitable base. Certain particularly preferred acid addition salts of the active agents herein include halide salts, such as may be prepared using hydrochloric or hydrobromic acids. Conversely, preparation of basic salts of the active agents of this invention are prepared in a similar manner using a pharmaceutically acceptable base such as sodium hydroxide, potassium hydroxide, ammonium hydroxide, calcium hydroxide, trimethylamine, or the like. Particularly preferred basic salts include alkali metal salts, e.g., the sodium salt, and copper salts.

For the preparation of salt forms of basic drugs, the pKa of the counterion is preferably at least about 2 pH units lower than the pKa of the drug. Similarly, for the preparation of salt forms of acidic drugs, the pKa of the counterion is preferably at least about 2 pH units higher than the pKa of the drug. This permits the counterion to bring the solution's pH to a level lower than the pHₘₐₓ to reach the salt plateau, at which the solubility of salt prevails over the solubility of free acid or base. The generalized rule of difference in pKa units of the ionizable group in the active pharmaceutical ingredient (API) and in the acid or base is meant to make the proton transfer energetically favorable. When the pKa of the API and counterion are not significantly different, a solid complex may form but may rapidly disproportionate (i.e., break down into the individual entities of drug and counterion) in an aqueous environment.

Preferably, the counterion is a pharmaceutically acceptable counterion. Suitable anionic salt forms include, but are not limited to acetate, benzoate, benzylate, bitartrate, bromide, carbonate, chloride, citrate, edetate, edisylate, estolate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate (embonate), phosphate and diphosphate, salicylate and disalicylate, stearate, succinate, sulfate, tartrate, tosylate, triethiodide, valerate, and the like, while suitable cationic salt forms include, but are not limited to aluminum, benzathine, calcium, ethylene diamine, lysine, magnesium, meglumine, potassium, procaine, sodium, tromethamine, zinc, and the like.

Preparation of esters typically involves functionalization of hydroxyl and/or carboxyl groups that are present within the molecular structure of the active agent. In certain embodiments, the esters are typically acyl-substituted derivatives of free alcohol groups, i.e., moieties that are derived from carboxylic acids of the formula RCOOH where R is alky, and preferably is lower alkyl. Esters can be reconverted to the free acids, if desired, by using conventional hydrogenolysis or hydrolysis procedures.

Amides can also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine.

In various embodiments, the active agents identified herein (e.g., combined GABA receptor activating ligand(s) and PAM(s)) are useful for parenteral administration, topical administration, oral administration, nasal administration (or otherwise inhaled), rectal administration, or local administration, such as by aerosol or transdermally, for promoting the replication, and/or survival, and/or function of beta cells in mammal.

In various embodiments the active agents described herein can also be combined with a pharmaceutically acceptable carrier(s) (excipient(s)) to form a pharmacological composition containing both agents. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, protection and uptake enhancers such as lipids, compositions that reduce the clearance or hydrolysis of the active agents, or excipients or other stabilizers and/or buffers.

Other physiologically acceptable compounds, particularly of use in the preparation of tablets, capsules, gel caps, and the like include, but are not limited to binders, diluent/fillers, disintegrants, lubricants, suspending agents, and the like.

In certain embodiments, to manufacture an oral dosage form (e.g., a tablet), an excipient (e.g., lactose, sucrose, starch, mannitol, etc.), an optional disintegrator (e.g. calcium carbonate, carboxymethylcellulose calcium, sodium starch glycollate, crospovidone etc.), a binder (e.g. alpha-starch, gum arabic, microcrystalline cellulose, carboxymethylcellulose, polyvinylpyrrolidone, hydroxypropylcellulose, cyclodextrin, etc.), and an optional lubricant (e.g., talc, magnesium stearate, polyethylene glycol 6000, etc.), for instance, are added to the active component or components (e.g., alaproclate and other compounds described herein, or a tautomer(s) or stereoisomer(s) thereof, or pharmaceutically acceptable salts or solvates of said alaproclate and other compounds, said stereoisomer(s), or said tautomer(s), or analogues, derivatives, or prodrugs thereof) and the resulting composition is compressed. Where necessary the compressed product is coated, e.g., using known methods for masking the taste or for enteric dissolution or sustained release. Suitable coating materials include, but are not limited to ethyl-cellulose, hydroxymethylcellulose, POLYOX®yethylene glycol, cellulose acetate phthalate, hydroxypropylmethylcellulose phthalate, and Eudragit (Rohm & Haas, Germany; methacrylic-acrylic copolymer).

Other physiologically acceptable compounds include wetting agents, emulsifying agents, dispersing agents or preservatives that are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physiochemical characteristics of the active agent(s).

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

The pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectibles, implantable sustained-release formulations, mucoadherent films, topical varnishes, lipid complexes, etc.

Pharmaceutical compositions comprising the active agents described herein (e.g., GABA receptor activating ligand(s) and PAM(s)) can be manufactured by means of conventional mixing, dissolving, granulating, dragee-making, levigating, emulsifying, encapsulating, entrapping or lyophilizing processes. Pharmaceutical compositions can be formulated in a conventional manner using one or more physiologically acceptable carriers, diluents, excipients or auxiliaries that facilitate processing of the active agent(s) into preparations that can be used pharmaceutically. Proper formulation is dependent upon the route of administration chosen.

In certain embodiments, the combined active agents (e.g., GABA receptor activating ligand(s) and PAM(s)) are formulated for oral administration. Suitable formulations or oral administration can be readily prepared by combining the active agent(s) with pharmaceutically acceptable carriers suitable for oral delivery well known in the art. Such carriers enable the active agent(s) described herein to be formulated as tablets, pills, dragees, caplets, lizenges, gelcaps, capsules, liquids, gels, syrups, slurries, suspensions and the like, for oral ingestion by a patient to be treated. For oral solid formulations such as, for example, powders, capsules and tablets, suitable excipients can include fillers such as sugars (e.g., lactose, sucrose, mannitol and sorbitol), cellulose preparations (e.g., maize starch, wheat starch, rice starch, potato starch, gelatin, gum tragacanth, methyl cellulose, hydroxypropylmethyl-cellulose, sodium carboxymethylcellulose), synthetic polymers (e.g., polyvinylpyrrolidone (PVP)), granulating agents; and binding agents. If desired, disintegrating agents may be added, such as the cross-linked polyvinylpyrrolidone, agar, or alginic acid or a salt thereof such as sodium alginate. If desired, solid dosage forms may be sugar-coated or enteric-coated using standard techniques. The preparation of enteric-coated particles is disclosed for example in U.S. Pat. Nos. 4,786,505 and 4,853,230.

For administration by inhalation, the active agent(s) (e.g., GABA receptor activating ligand(s) and PAM(s) described herein) are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit can be determined by providing a valve to deliver a metered amount. Capsules and cartridges of e.g. gelatin for use in an inhaler or insufflator may be formulated containing a powder mix of the compound and a suitable powder base such as lactose or starch.

In certain embodiments the active agents described herein are formulated for systemic administration (e.g., as an injectable) in accordance with standard methods well known to those of skill in the art. Systemic formulations include, but are not limited to, those designed for administration by injection, e.g. subcutaneous, intravenous, intramuscular, intrathecal or intraperitoneal injection, as well as those designed for transdermal, transmucosal oral or pulmonary administration. For injection, the active agents described herein can be formulated in aqueous solutions, preferably in physiologically compatible buffers such as Hanks solution, Ringer's solution, or physiological saline buffer and/or in certain emulsion formulations. The solution(s) can contain formulatory agents such as suspending, stabilizing and/or dispersing agents. In certain embodiments the active agent(s) can be provided in powder form for constitution with a suitable vehicle, e.g., sterile pyrogen-free water, before use. For transmucosal administration, and/or for blood/brain barrier passage, penetrants appropriate to the barrier to be permeated can be used in the formulation. Such penetrants are generally known in the art. Injectable formulations and inhalable formulations are generally provided as a sterile or substantially sterile formulation.

In addition to the formulations described previously, the active agents (e.g., GABA receptor activating ligand(s) and PAM(s) described herein) may also be formulated as a depot preparations. Such long acting formulations can be administered by implantation (for example subcutaneously or intramuscularly) or by intramuscular injection. Thus, for example, the active agent(s) may be formulated with suitable polymeric or hydrophobic materials (for example as an emulsion in an acceptable oil) or ion exchange resins, or as sparingly soluble derivatives, for example, as a sparingly soluble salt.

In certain embodiments the active agent(s) described herein can also be delivered through the skin using conventional transdermal drug delivery systems, i.e., transdermal “patches” wherein the active agent(s) are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active ingredient(s)” that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one illustrative embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the “patch” and provides the device with much of its flexibility. The material selected for the backing layer is preferably substantially impermeable to the active agent(s) and any other materials that are present.

Alternatively, other pharmaceutical delivery systems can be employed. For example, liposomes, emulsions, and microemulsions/nanoemulsions are well known examples of delivery vehicles that may be used to protect and deliver pharmaceutically active compounds. Certain organic solvents such as dimethylsulfoxide also can be employed, although usually at the cost of greater toxicity.

In certain embodiments the combined formulation comprises the GABA receptor activating ligand(s) and/or the PAM(s) at a dosage (unit dosage) than would be provided if the GABA receptor activating ligand and/or the PAM were used alone. In certain embodiments GABA receptor activating ligand is present at less than about 90%, or less than about 80%, or less than about 70%, or less than about 60%, or less than about 50%, or less than about 40%, or less than about 40%, or less than about 30%, or less than about 10%, or less than about 5% of the GABA receptor activating ligand required to promote the replication, and/or survival, and/or function of beta cells when the GABA receptor activating ligand is used alone.

In certain embodiments the PAM(s) are provided at a subtherapeutic dosage. This refers to a dosage below the approved and/or recommended and/or recognized dosage of the PAM when used for the activity for which the PAM was originally designed and/or approved.

Thus, for example, the recommended/approved dosage of alprazolam (immediate release tablets) for treatment of panic disorder is an initial dose of 0.5 mg orally 3 times a day with a maintenance dose of 1 to 10 mg/day in divided doses and a mean dose of 5 to 6 mg/day in divided doses. The recommended/approved dosage extended release alprazolam tablets is 0.5 mg to 1 mg once a day and the maintenance dose is 1 to 10 mg once a day with a mean dose of 3 to 6 mg once a day. The recommended/approved dosage of alprazolam (immediate release tablets) for treatment of adult depression is an initial dose of 0.5 mg orally 3 times a day which is increased to an average effective dose of 3 mg orally daily in divided doses. Geriatric doses for anxiety are provided at an initial dose of 0.25 mg orally 2 to 3 times a day in elderly or debilitated patients and a daily dose greater than 2 mg meets the Beers criteria as a medication that is potentially inappropriate for use in older adults.

In a formulation a subtherapeutic dosage is typically less than the lowest recommended/approved unit dosage form, e.g., in the case of alprazolam less than 0.25 mg as a unit dosage form and a total of less than 0.5 mg daily in a treatment method. In certain embodiments the subtherapeutic dosage is less than 90% or less than about 80%, or less than about 70%, or less than about 60%, or less than about 50%, or less than about 40%, or less than about 30%, or less than about 20%, or less than about 10% of this recommended/approved dosage.

These dosages are intended to be illustrative and not limiting. The actual dosage amount of the BABA receptor activating ligand and PAM administered to a subject can be determined by physical and physiological factors such as body weight, severity of condition, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

### Kits.

In certain embodiments kits are provided for the practice of the methods described herein. In various embodiments the kits comprise a container containing a GABA activating ligand, and a container containing positive allosteric modulator of the GABA_{A} receptor as described herein. In certain embodiments the kit comprises GABA and alprazolam.

In certain embodiments the GABA receptor activating ligand(s) and PAM(s) can be provided in unit dosage formulation(s) (e.g., tablet, caplet, patch, etc.) and/or may be optionally combined with one or more pharmaceutically acceptable excipients. In certain embodiments the GABA receptor activating ligand(s) and PAM(s) can be provided in separate containers. In certain embodiments the GABA receptor activating ligand(s) and PAM(s) can be provided in the same container. In certain embodiments the GABA receptor activating ligand(s) and PAM(s) can be provided in the same container as a combined formulation.

In addition, the kits optionally include labeling and/or instructional materials providing directions (i.e., protocols) for the practice of the methods of this invention. Certain labeling or instructional materials describe the use of a combination of GABA receptor activating ligand(s) and PAM(s) to promote the replication, and/or survival, and/or function of beta cells in mammal (e.g., a mammal receiving an islet cell transplant). The labeling or instructional materials may also, optionally, teach preferred dosages/therapeutic regiment, counter indications and the like.

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### Use of a Gaba Receptor Activating Ligand in Combination with Alprazolam to Enhance Beta Cell Replication

In this example we studied combination of the benzodiazepine alprazolam (XANAX®) in with GABA for its effect on beta cell proliferation and/or survival. Alprazolam is widely prescribed for the treatment of anxiety and panic disorders. After its introduction in 1981 it rapidly became a blockbuster drug, with over 50 million prescriptions. When used as directed, it is safe for long term use (see FDA approved labeling at www.accessdata.fda.gov/drugsatfda_docs/label/2011/018276s0451bl.pdf) and (Jonas and Cohon (1993) J. Clin. Psychiatry. 54 Suppl:25-45; discussion 6-8)). We chose to study alprazolam based on 1) its long-term safety profile, and 2) its interaction with the appropriate GABAA-R subunits.

### GABA Receptor Activating Ligand.

GABA is safe for human consumption. GABA is sold over the counter as a nutraceutical. We observed that GABA treatment for 26 weeks did not significantly alter the total numbers of splenic mononuclear cells or percentages of CD4₊, CD8₊, T and B lymphocytes (Tian et al. (2004) J. Immunol. 173(8): 5298-5304), nor did it desensitize autoreactive T cells to GABA-mediated inhibition (Tian et al. (2004) J. Immunol. 173(8): 5298-5304; Tian (2011) Autoimmunity, 44: 465-470). Long-term oral GABA treatment was tested in the 1950s-1980s in human clinical trials for its ability to reduce epileptic seizures and ameliorate cerebrovascular disorders in hundreds of individuals (Otomo et al. (1981) Arzneimittelforschung. 31(9): 1511-1523; Loeb et al. (1987) Epilepsy Res. 1(3): 209-212; Tower and Roberts eds. Inhibition in the Nervous System and GABA. New York: Pergamon Press, 1960; Kuriyama and Sze (1971) Neuropharmacol. 10(1): 103-108). The results showed no adverse effects, but no clinical benefit, which may be because GABA does not cross the blood-brain barrier.

Pharmaceutical interests have focused on drugs that can get through the blood-brain barrier and modulate GABARs on CNS neurons to ameliorate seizure disorders, anxiety and insomnia. The inability of GABA to get through the blood-brain barrier makes it ideal to modulate peripheral GABA-Rs without CNS side effects. GABA's ability to downregulate inflammatory responses is modest, which we view as being advantageous because there is less interference with immune system function and no leukopenia as with other immunosuppressants. Despite GABA's mild effects, it efficiently prevented T1D (Tian et al. (2004) J. Immunol. 173(8): 5298-5304: Soltani et al. (2011) Proc. Natl. Acad. Sci. USA, 108: 11692-11697), EAE (Bhat (2010) Proc. Natl. Acad. Sci. USA, 107(6): 2580-2585) and rheumatoid arthritis (Tian (2011) Autoimmunity, 44: 465-470), and ameliorated T2D (Tian et al. (2011) PloS one. 6(9): e25338) in mice with different genetic backgrounds.

GABA_{A}-Rs and GABA_{B}-Rs.

There are two different types of GABA-Rs; GABA_{A}-Rs and GABA_{B}-Rs. Immune cells primarily express GABA_{A}-Rs (Tian et al. (1999) J. Neuroimmunol. 96(1): 21-28; Tian et al. (2004) J. Immunol. 173(8): 5298-5304; Bhat (2010) Proc. Natl. Acad. Sci. USA, 107(6): 2580-2585; Bjurstom et al. (2008) J. Neuroimmunol. 205: 44-50) while β-cells express both GABA_{A}-Rs and GABA_{B}-Rs (Ligon et al. (1997) Diabetologia, 50(4): 764-773; Tian et al. (2013) Diabetes, 62(11): 3760-3765; Braun et al. (2010) Diabetes, 59(7): 1694-2701; Gu et al. (1993) Life Sci. 52(8): 687-694; Brice et al. (2002) Diabetologia, 45(2): 242-252; Martin et al. (1988) Neuropsychobiology, 19(3): 146-148). GABA_{A}-Rs are pentamers of different subunits that form fast-acting chloride channels. GABA_{B}-Rs are heterodimers of just two subunits, but their properties can be altered by other cellular proteins (Schwenk et al. (2010) Nature, 465(7295): 231-235). GABA_{B}-Rs form a slow-acting G-protein coupled receptor (Bettler et al. (2004) Physiol. Rev. 84(3): 835-867). Thus, GABA_{A}-Rs and GABA_{B}-Rs are quite different (a chloride channel vs. a G-protein coupled receptor). Our focus in this example is on GABA_{A}-Rs because they have the capability to simultaneously inhibit autoimmune responses and promote β-cell replication and survival.

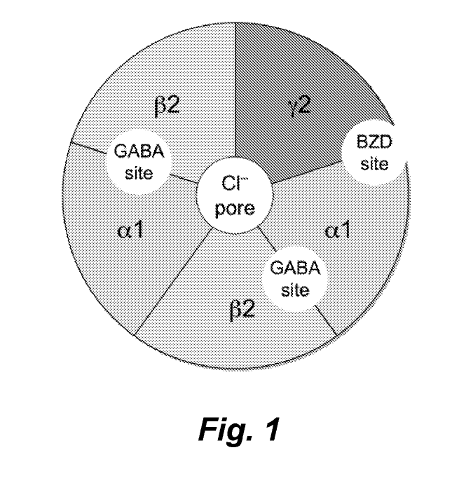

There are 19 different GABA_{A}-R subunits: six types of a subunits, three different β subunits, three γ's, as well as a δ, ε, π, and θ subunit, plus three nonclassical ρ subunits. Five of the subunits combine in different ways to form GABA_{A} channels that usually consist of two α's, two β's, and one of the other subunit types (see schematic diagram in FIG. 1). GABA_{A}-Rs with different subunit combinations have different pharmacological properties and specific agonists and antagonists (Olsen and Tobin (1990) FASEB J. 4(5): 1469-1480; Luddens et al. (1995) Neuropharmacology, 34(3): 245-254). In β-cells, the activation of the GABA_{A}-R Cl-channel causes depolarization, the opening of voltage-dependent calcium channels, and the activation of Ca₂₊-dependent Pi3K/Akt cell growth and survival signaling pathway (Soltani et al. (2011) Proc. Natl. Acad. Sci. USA, 108: 11692-11697; Purwana et al. (2014) Diabetes, 63(12):4197-4205; Braun et al. (2010) Diabetes, 59(7): 1694-2701).

### Benzodiazepines.

Benzodiazepines do not bind to the GABA-binding site, but rather elsewhere on GABA_{A}-Rs (the “BZD” site in diagram). While they cannot open the chloride channel, they act as positive allosteric modulators that increase in Cl-conductance when GABA is bound to the receptor. Unlike GABA, which has little or no ability to pass through the blood-brain barrier, benzodiazepines can pass through the blood-brain barrier and enhance the action of GABA produced by CNS neurons. Different benzodiazepines preferentially bind to different GABA_{A}-Rs with different subunit compositions (subtypes).

Benzodiazepines with different GABA_{A}-R subtype specificity have been used to ameliorate seizures, insomnia, or anxiety. In this example, we study the benzodiazepine alprazolam (XANAX®). Alprazolam is widely prescribed for the treatment of anxiety and panic disorders. After its introduction in 1981 it rapidly became a blockbuster drug, with over 50 million prescriptions. When used as directed, it is safe for long term use (see FDA approved labeling at www.accessdata.fda.gov/drugsatfda_docs/label/2011/018276s0451bl.pdf and (Jonas and Cohon (1993) J. Clin. Psychiatry. 54 Suppl:25-45; discussion 6-8)). We chose to study alprazolam based on 1) its long-term safety profile, 2) the appropriate GABA_{A}-R subunits are expressed in human islets (Braun et al. (2010) Diabetes, 59(7): 1694-2701) and 3) alprazolam's ability to reduce HbA1C levels in individuals with poor glucose control (Lustman et al. (1995) Diabetes Care, 18(8): 1133-1139, see below).

Alprazolam Reduced HbA1c in Patients with Poor Glucose Control.

We hypothesized that a benzodiazepine that binds to the particular GABA_{A}-R subtype(s) expressed by human β-cells could enhance the action of GABA that is locally released within the islets. In a clinical study, alprazolam was administered to anxious and non-anxious individuals with poor glucose control (HbA1c average of about 12) with T1D or T2D. After 8 weeks of treatment, HbA1c levels were significantly reduced in patients that received alprazolam but not placebo (Lustman et al. (1995) Diabetes Care, 18(8): 1133-1139). This effect was similar in both anxious and non-anxious patients indicating that the reduction in HbA1c was not dependent upon improvement of anxiety.

### Alprazolam Promotes Human Islet Cell Proliferation In Vitro.

In pilot studies, we tested whether alprazolam alone, and in combination with low levels of GABA, could enhance human islet cell replication in vitro. We tested alprazolam alone because although benzodiazepines themselves cannot open the GABA_{A}-R chloride channel, the β-cells express GAD and secrete GABA. Accordingly, we hypothesized that alprazolam may enhance the actions of endogenously produced islet GABA.

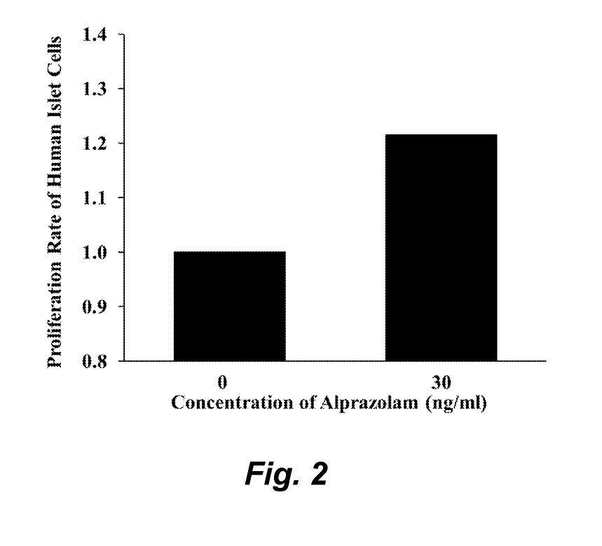

Alprazolam is prescribed in extended release tablets that deliver 0.5-6.0 mg/day and its concentration is usually in a range of 10-100 ng/ml in the serum of persons receiving the drug therapeutically (Jones et al. (2007) Therap. Drug Monitor. 29(2): 248-360; Fraser and Bryan (1991) J. Analyt. Toxicol., 15(2): 63-65). We cultured human islets with, or without, 30 ng/ml alprazolam. We found that alprazolam promoted human islet cell proliferation by about 123% compared to cultures with no alprazolam (FIG. 2). Since we did not add GABA to these cultures, it is likely that alprazolam acted with GABA secreted from β-cells to promote islet cell proliferation. In this case, the GABA that was secreted from the 50 cultured islets/well probably diffused away very quickly and limited the effectiveness of alprazolam, a notion that is supported by the results of our study in which GABA was added to the culture medium (see below).

In patients newly diagnosed with T1D, β-cell mass is greatly reduced, such that the levels of secreted GABA within the islet should be low. We postulated that remaining GABA secretion along with alprazolam may enhance GABA's actions on residual β-cell replication and survival and may explain how alprazolam decreased HbA1C levels in patients with T1D and T2D. Notably, we have shown that GABA monotherapy can enhance β-cell replication and survival in newly diabetic NOD mice (Soltani et al. (2011) Proc. Natl. Acad. Sci. USA, 108: 11692-11697; Tian et al. (2014) Diabetes. 63(9): 3128-3134).

Our hypothesis tested in this study was that the ability of residual β-cells to secrete GABA is suboptimal and that exogenous GABA treatment is beneficial for promoting β-cell replication and survival in newly diabetic mice. Moreover, we further tested whether combination treatment with GABA and alprazolam has enhanced ability to promote β-cell replication and survival and/or can reduce the amount of either drug needed to achieve these effects.

### Alprazolam and GABA Act Synergistically to Promote Human Islet Cell Replication at Reduced Dosage In Vitro.

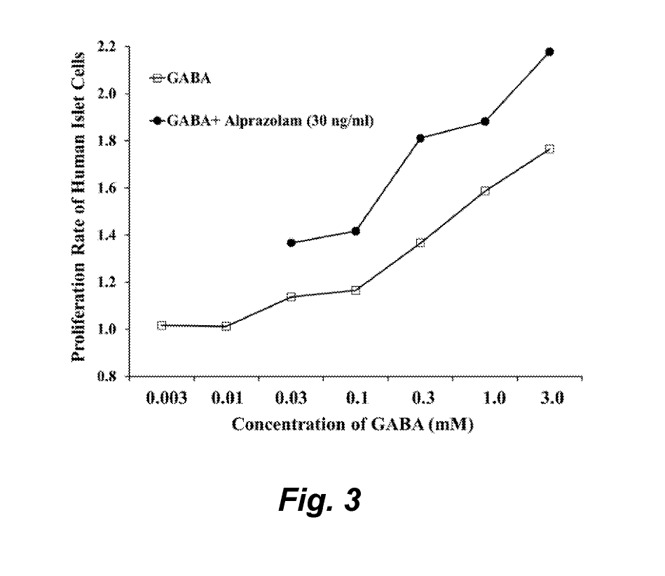

We next compared human islet cell proliferation in human islet cultures that contained a dose range of GABA with, or without, alprazolam (30 ng/ml). While treatment with 0.03 mM GABA did not significantly enhance human islet cell proliferation treatment, the same dose of GABA together with alprazolam enhanced human islet cell proliferation by 138% (FIG. 3). To achieve a similar level of proliferation using GABA alone required a 10-fold higher level of GABA (0.3 mM). Thus, alprazolam greatly reduces the amount of GABA needed to induce fairly robust β-cell proliferation. These data demonstrate that the combination of GABA and alprazolam has a synergistic positive effect on human islet cell proliferation in vitro.

### Combined Alprazolam GABA Act Synergistically to Inhibit β-Cell Autoimmunity.

An additional advantage of administering GABA along with alprazolam is the potential for synergistically inhibiting autoreactive T cells and promoting Tregs. The level of GABA in the blood is very low (there is very little GAD outside the CNS to produce GABA, and GABA has a very short half-life in circulation). Without GABA, alprazolam alone should not have much effect on immune cells, but in combination they may act synergistically to inhibit inflammation and promote Tregs, perhaps using reduced dosages of each drug. While studies of alprazolam+GABA's effects on immune responses are of great interest, they are outside the scope of this study, which focused on human β-cell replication. Translatability of alprazolam for enhancing β-cell replication and survival in humans. Alprazolam does not cause immune suppression or increase susceptibility to infections in humans (see the alprazolam fact sheet at http://labeling.pfizer.com/ShowLabeling.aspx?id=547) consistent with the notion that without GABA, alprazolam cannot activate GABA-Rs on human immune cells. Alprazolam's main side effect is sleepiness.

After long-term alprazolam use, withdrawal and rebound symptoms commonly occur and necessitate a gradual reduction in dosage (Verster and Volkerts (2004) CNS Drug Rev., 10(1): 45-76). In rodent studies, alprazolam inhibited EAE and inflammation in the spinal cords of stressed rats (Nunez-Iglesias et al. (2010) Pharmacol. Biochem., Behav. 97(2): 350-356), which is interesting because alprazolam may have enhanced the actions of neuronally produced GABA on infiltrating immune cells. In this case, it may have similar beneficial actions on islet-infiltrating immune cells. The only other reports of alprazolam affecting the immune system were 1) one group reported that alprazolam inhibited the proliferative responses of mouse lymphocytes and reduced the production of IL-2 by T cells and TNF by macrophages (Chang et al. (1991) Int. J. Pharmacol., 13(2-3): 259-266; Chang et al. (1992) Int. J. Pharmacol., 14(2): 227-337), and 2) in direct contrast, another group reported that alprazolam enhanced mitogen-induced lymphocyte proliferation and NK cell activity (Fride et al. (1990) Life Sci., 47(26): 2409-2420).

Alprazolam should not be confused with other benzodiazepines such as diazepam (valium) and lorazepam that have adverse side effects on the immune system and the CNS. We are unaware of any reports that alprazolam binds significantly to the protein previously known as the “peripheral benzodiazepine receptor”, which is now known to be a translocator protein (TSPO) on the outer mitochondrial membrane (Papadopoulos et al. (2006) Trend. Pharmacol. Sci. 27(8): 402-409), and is a secondary binding site for diazepam.

As noted above, alprazolam significantly reduced HbA1c in patients with poor glucose control (Lustman et al. (1995) Diabetes Care, 18(8): 1133-1139). Our pilot studies show that a modest level of alprazolam (alone) had a fairly robust effect on human islet cell proliferation in vitro (FIG. 2). The combination of alprazolam and GABA had a synergistic effect on human islet cell proliferation such that a ten-fold higher level of GABA (alone) was needed to achieve a similar proliferative effect (FIG. 3). This suggests that in combination with GABA, lower levels of alprazolam may promote β-cell replication and survival. Since alprazolam need not get through the blood brain barrier, lower dosages of alprazolam than those used for anxiolytic (anti-anxiety) effects may be effective for modulating β-cell GABAA-Rs in the periphery. Additionally, in the CNS, GABA_{A}-Rs are clustered together on neuronal synapses while extra-synaptic GABA-Rs have a diffuse distribution (Craig et al. (1994) Proc. Natl. Acad. Sci. USA, 91(26): 12373-12377). It is believed that clustered GABA_{A}-Rs have a 3-4 fold higher EC₅₀ for GABA and deactivate faster compared to diffuse extra-synaptic GABA-Rs because of a decrease in affinity for GABA (Chen et al. (2000) Proc. Natl. Acad. Sci. USA, 97(21): 11557-11562). It is thought that protein-protein interactions between clustered GABA-Rs affect the binding site or channel activation (Chen et al. (2000) Proc. Natl. Acad. Sci. USA, 97(21): 11557-11562). Without being bound to a particular theory, we believe it is likely that GABA_{A}-Rs on β-cells are diffuse and more sensitive to GABA and allosteric modulation.

It should be emphasized that promoting β-cell regeneration and health may be of little use if the autoimmune response against β-cells cannot be significantly curtailed. In this regard, the proposed alprazolam+GABA combination therapy is believed to excel due to its ability to simultaneously inhibit autoreactive T cells and promote Tregs. Notably, anxiolytic doses of alprazolam were able to decrease HbA1c in diabetic patients (Lustman et al. (1995) Diabetes Care, 18(8): 1133-1139). We contend that even if anxiolytic doses of alprazolam are required to promote β-cell survival and replication, the benefits of short-term alprazolam treatment outweigh the side effects (primarily sleepiness). For example, in the case of islet transplantation, alprazolam treatment would be limited to the period shortly after islet implantation in order to reduce β-cell loss due to hypoxia and stress. In the case of new onset T1D, alprazolam treatment may be given short-term in combination with GABA and, for example, anti-CD3—this might synergistically inhibit autoimmunity and induce Tregs, allowing C-peptide levels to be preserved for a longer time period than after treatment with anti-CD3 alone.

### Example 2

### Testing the Effect of Midazolam and Clonazepam on INS-1 Cell Proliferation In Vitro

The objective of this experiment was to test the effect of the following compounds on INS-1 cell proliferation in vitro:

Midazolam (MW 325): soluble in liquid at 6.25 mM; and

Clonazepam (MW315): dissolved in ethanol at 50 mM.

### Methods:

1. ³H-thymidine incorporation assay: INS-1 cells at 1×10⁵/well were treated in triplicate with indicated concentrations of individual compounds and cultured in 10% FCS RPMI164 medium in the presence of 0.3 μCi/well of 3H-thymidine for 48 hours (an optimal time period). The cells were harvested and the levels of ³H-thymidine uptake in individual wells were measured by beta-counter.

2. MTT assay: INS-1 cells at 1×10⁵/well were treated in quadruplicate with the indicated compounds in 10% FCS RPMI164 medium (phenol-free) for 44 hours. The cells were exposed to 20 ml of MTT (20 mg/ml) for 4 hours and the supernatants of cultured cells were removed. The resulting products were dissolved in 100 ml DMSO and measured at absorbance of 540/650 nm in microplate reader.

### Results:

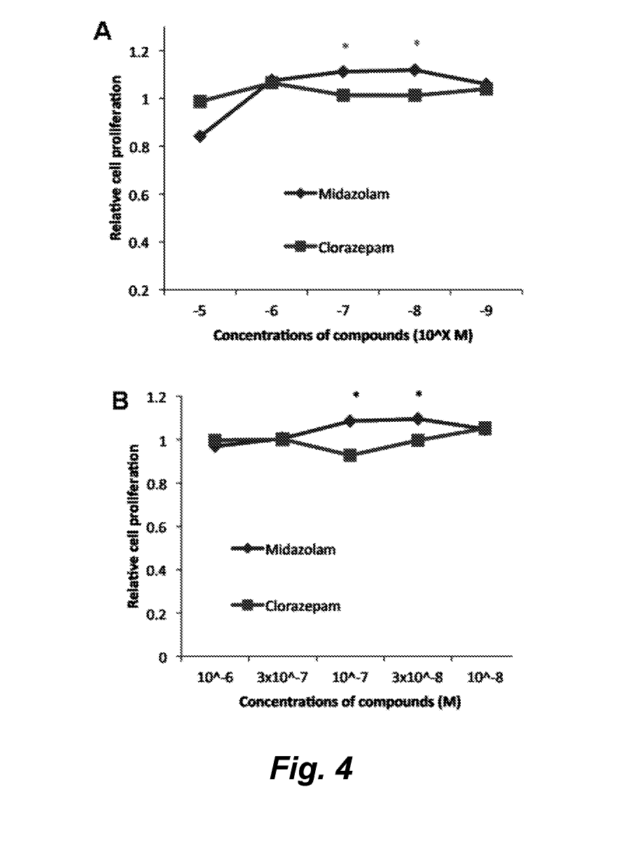

1. Treatment with midazolam, but not clonazepam, has some ability to enhance the proliferation of INS-1 cells in vitro. We found that treatment with clonazepam at 10⁻⁴-10⁻⁹ M did not significantly change the proliferation of INS-1 cells. Treatment with midazolam at 10⁻⁵ M had a slight toxicity against INS-1 cells and treatment with 10⁻⁷-10⁻⁸ M significantly enhanced INS-1 cell proliferation in vitro (FIG. 4A, p<0.05). Based on those results, we next tested these drugs over a smaller dose range with additional intermediate dosages and observed that midazolam at 10⁻⁷-3×10⁻⁸ M significantly enhanced the proliferation of INS cells in vitro (FIG. 4B). A similar pattern of cell proliferation was observed by MTT assays (data not shown).

2. Treatment with midazolam or clonazepam enhances GABA stimulated INS-1 cell proliferation in vitro. Next, we tested the effect of midazolam and clonazepam on GABA-induced INS-1 cell proliferation in vitro. We found that treatment with GABA at 1-0.1 mM stimulated INS-1 cell proliferation in vitro. Treatment with midazolam at 10⁻⁷ or 3×10⁻⁸ M enhanced GABA-induced INS-1 cell proliferation. While treatment with GABA at 0.03-0.01 mM failed to induce INS-1 cell proliferation treatment with both GABA at such low doses and midazolam at 10⁻⁷ or 3×10⁻⁸ M significantly enhanced INS-1 cell proliferation. The degree of INS-1 cell proliferation in the cells treated with both low doses of GABA and midazolam were higher than that in the cell treated with midazolam alone, indicating that midazolam potentiated GABA-induced cell proliferation. Similarly, treatment with clonazepam at 10⁻⁷ or 3×10⁻⁸ M significantly enhanced GABA-stimulated INS-1 cell proliferation in vitro. A similar pattern of pharmacological effect of these drugs on GABA-induced cell proliferation was detected by MTT assays.

The enhanced effect of midazolam or clonazepam is not mediated by translocator protein (18 kDa) (TSPO) which can bind some benzodiazepines.

To test whether the effects of midazolam and clonazepam were mediated in part through TSPO, we tested the impact of TSPO inhibitor of PK11195 on midazolam or clonazepam enhanced GABA-induced INS-1 cell proliferation. We found that treatment with the indicated GABA or midazolam, but not clonazepam or PK11195 induced INS-1 cell proliferation. Treatment with both GABA and midazolam, or GABA and clonazepam, enhanced cell proliferation, which was not affected by PK11195. These data indicated that the enhanced proliferation of INS-1 cells by midazolam or clonazepam was not mediated by activating TSPO in INS-1 cells.

### Example 3

### Testing the Effect of AP325 and AP3 on INS-1 Cell Proliferation In Vitro

The objective of this experiment was to test the effect of the following compounds on INS-1 cell proliferation in vitro:

AP325 (MW 310): soluble in DMSO at 50 mM; and

AP3 (MW 276.33): dissolved in DMSO at 50 mM.

### Methods:

1. ³H-thymidine incorporation assay: INS-1 cells at 1×10⁵/well were treated in triplicate with indicated concentrations of individual compounds and cultured in 10% FCS RPMI164 medium in the presence of 0.3 uCi/well of ³H-thymidine for 48 hours (an optimal time period). The cells were harvested and the levels of ³H-thymidine uptake in individual wells were measured by beta-counter.

2. MTT assay: INS-1 cells at 1×10⁵/well were treated in quadruplicate with the indicated compounds in 10% FCS RPMI164 medium (phenol-free) for 44 hours. The cells were exposed to 20 ml of MTT (20 mg/ml) for 4 hours and the supernatants of cultured cells were removed. The resulting products were dissolved in 100 ml DMSO and measured at absorbance of 540/650 nm in a microplate reader.

### Results:

### Treatment with AP325 or AP3 (Alone) Does Not Stimulate the Proliferation of INS-1 Cells In Vitro

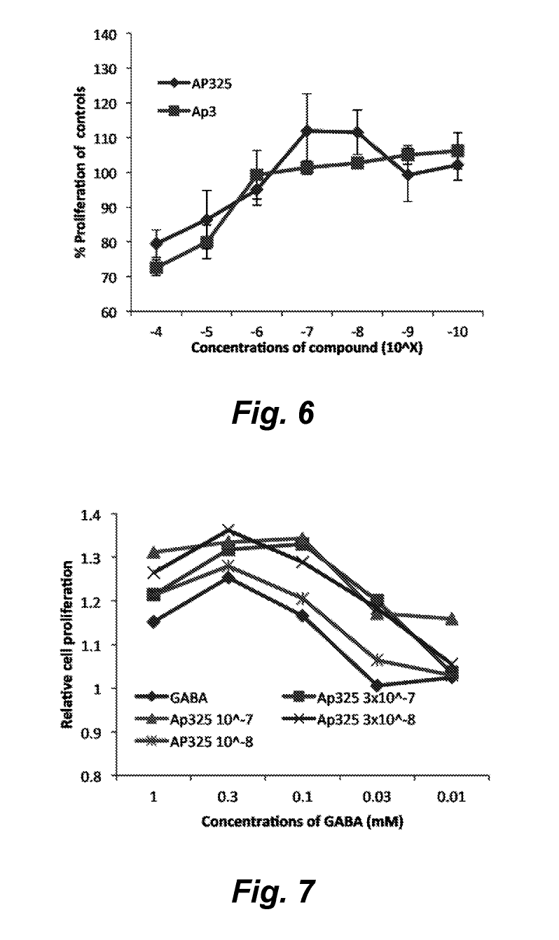

We found that treatment with AP325 or AP3 at 10⁻⁴-10⁻⁵ M significantly inhibited INS-1 cell proliferation in vitro (FIG. 6). Although treatment with either compound at 10⁻⁶-10⁻¹⁰ did not inhibit the proliferation of INS-1 cells the treatment did not increase significantly the proliferation of INS-1 cells. Notably, treatment with AP325, but not AP3, at 10⁻⁷-10⁻⁸ slightly increased the proliferation of INS-1 cells (but this was nonsignificant). A similar pattern of cell survival was detected by MTT assay (data not shown). Hence, both compounds at high dose had toxicity against INS-1 cells and those compounds at 10⁻⁶-10⁻¹⁰ did not show any stimulation of INS cell proliferation in vitro.

### Treatment with AP325 Enhances GABA-Stimulated INS-1 Cell Proliferation In Vitro

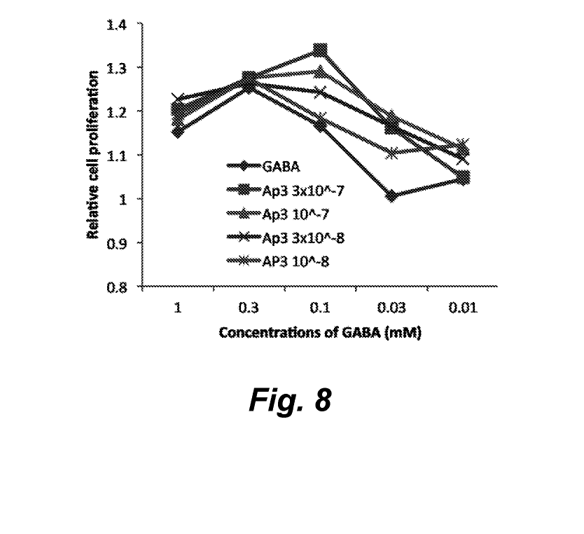

Next, we tested the effect of AP325 or AP3 on GABA-induced INS-1 cell proliferation in vitro. We found that treatment with GABA at 1-0.1 mM stimulated INS-1 cell proliferation in vitro, but treatment with GABA 0.03 or 0.01 mM did not significantly stimulate INS-1 cell proliferation in vitro (FIG. 7). Treatment with AP325 at 10⁻⁶ M did not significantly change the proliferation of INS-1 cells (data not shown). However, treatment with AP325 10⁻⁷-10⁻⁸ M significantly enhanced GABA-induced INS-1 cell proliferation, particularly with AP325 at 3×10⁻⁷-3×10⁻⁸ M. Notably, while treatment with GABA at 0.03 or 0.01 mM alone failed to stimulate INS-1 cell proliferation in our experimental condition, treatment with AP325 at 3×10⁻⁷-10⁻⁸ M, together with 0.03 mM GABA significantly promoted INS-1 cell proliferation. Furthermore, treatment with AP325 at 10⁻⁷ together with 0.01 mM GABA also significantly stimulated INS-1 cell proliferation in vitro. Similar patterns of data were obtained from MTT assays (data not shown). Therefore, these data indicated that AP325 enhanced GABA stimulated INS-1 cell proliferation in vitro.

### Treatment with AP3 Enhances GABA-Stimulated INS-1 Cell Proliferation In Vitro

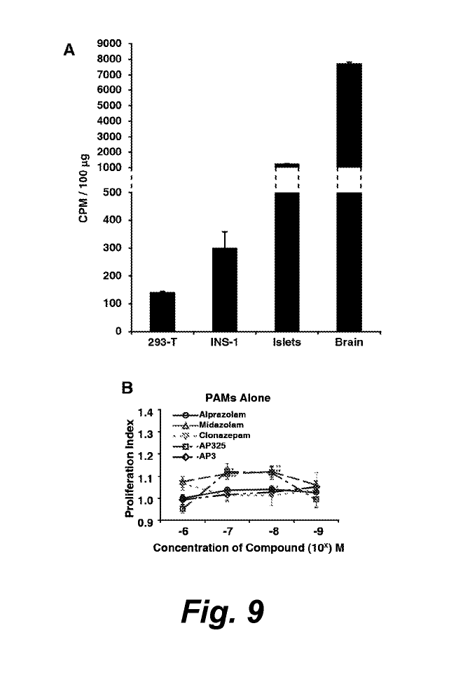

Analysis of AP3 revealed that treatment with AP3 at 10⁻⁶ M did not significantly change the proliferation of INS-1 cells (data not shown). However, treatment with AP3 at 3×10⁻⁷-3×10⁻⁸ M significantly enhanced 0.1 mM GABA-induced INS-1 cell proliferation (FIG. 8). Notably, treatment with AP3 at 3×10⁻⁷-10⁻⁸ M, together with 0.03 mM GABA significantly promoted INS-1 cell proliferation. Furthermore, treatment with AP3 at 10⁻⁷-10⁻⁸, together with 0.01 mM GABA, also significantly stimulated INS-1 cell proliferation in vitro. Similar patterns of data were obtained from MTT assays (data not shown). Therefore, these data indicated that AP3 enhanced GABA stimulated INS-1 cell proliferation in vitro.

### Conclusion:

AP325 and AP3 can enhance GABA-induced INS-1 cell proliferation in vitro.

### Example 4

### Clinically Applicable GABA Receptor Positive Allosteric Modulators Can Promote β-Cell Replication

A key goal of type 1 diabetes (T1D) research is to develop treatments to safely promote human β-cell replication. It has recently become appreciated that activation of γ-aminobutyric acid receptors (GABA-Rs) on β-cells can promote their survival and replication. A number of positive allosteric modulators (PAMs) that enhance GABA's actions on neuronal GABA_{A}-Rs are in clinical use. Repurposing these GABA_{A}-R PAMs to help treat diabetes is theoretically appealing because of their safety and potential to enhance the ability of GABA, secreted from β-cells, or exogenously administered, to promote β-cell replication and survival. Here, we show that clinically applicable GABA_{A}-R PAMs can enhance INS-1 β-cell replication and that this is amplified by exogenous GABA application. Furthermore, a GABA_{A}-R PAM promoted human islet cell replication in vitro. This effect was ablated by a GABA_{A}-R antagonist, suggesting that GABA released from β-cells can have an autocrine pro-mitotic effect that can be enhanced by a PAM. The combination of a PAM and low levels of exogenous GABA had increased ability to promote human β-cell replication. These studies identify a potential new class of drugs for T1D treatment and may explain past observations of a GABA_{A}-R PAM reducing HbA1c in diabetic patients.

### Introduction

A goal of T1D research is to discover approaches that can safely promote β-cell replication. However, few agents have been found that can enhance human β-cell replication. Rodent and human β-cells have been long known to express GABA-Rs, both GABA_{A}-Rs and GABA_{B}-Rs (Braun et al. (GABA) (2010) Diabetes, 59: 1694-1701), but only recently has it been shown that their activation can promote β-cell survival, replication and mass (Ligon et al. (2007) Diabetologia, 50: 764-773; Soltani et al. (2011) Proc. Natl. Acad. Sci. USA, 108: 11692-11697; Tian et al. (2013) Diabetes, 62: 3760-3765; Prud'homme et al. (2103) Transplantation, 96(7): 616-623; Purwana et al. (2014) Diabetes, 63(12): 4197-4205). GABA_{A}-R PAMs enhance the action of GABA. They do not bind to the GABA-binding site, but rather elsewhere on GABA_{A}-Rs and while they cannot open the GABA_{A}-R chloride channel, they increase in Cl⁻ conductance when GABA is bound to the receptor. Because GABA has little to no ability to pass through the blood brain barrier (BBB), BBB-permeable GABA_{A}-R PAMs, such as the benzodiazepines, were developed to enhance the action of GABA secreted by CNS neurons in order to treat CNS disorders such as seizures, insomnia, and anxiety. Theoretically, these PAMs could be repurposed to enhance the action of GABA that is released within the islets to promote β-cell mitogenesis.

Here, we tested clinically-applicable GABA_{A}-R PAMs for their ability to promote β-cell replication. Specifically, we tested alprazolam, midazolam, and clonazepam, which represent different classes of benzodiazepines, for their ability to promote replication of the β-cell line INS-1. Alprazolam has been widely used for treating anxiety and nearly 50 million prescriptions are written for this medication per year. It is safe for long-term use when used as directed (Jonas et al. (1993) J. Clin. Psychiatry, 54 Suppl: 25-45; discussion 46-28) and http://www.fda.gov/safety/medwatch/safetyinformation/ucm271398.htm). In addition, we tested a newly developed non-benzodiazepine GABA_{A}-R PAM, AP3, that is peripherally-restricted. We then examined the effect of alprazolam, with, or without, exogenous GABA, on human islet cell replication in vitro.

### Research Design and Methods

Alprazolam, midazolam, clonazepam, PK11195, and bicuculline were purchased from Sigma-Aldrich, and AP3 was provided by Algiax Pharmaceuticals GmbH. Stock solutions of alprazolam (10 mM in DMSO), midazolam (6.25 mM in water), clonazepam (50 mM in ETOH), or AP3 (50 mM in DMSO) were diluted into media to the indicated concentration.

Proliferation Assays.

INS-1 cells at 1×10⁵/well were treated in triplicate with indicated concentrations of individual compounds and cultured in 10% FCS RPMI164 medium in the presence of ³H-thymidine (0.3 μCi/well) for 48 hours (an optimal time period). The levels of ³H-thymidine uptake in individual wells were measured by a scintillation counter.

Fresh human islets were obtained from the Integrated Islet Distribution Program and islets (50-75 IEQ/well) were treated with the indicated dosage of GABA, together with, or without, the indicated PAM for 4 days in the presence of ³H thymidine (0.2 μCi/well).

### Results

INS-1 Cells Express Benzodiazepine-Binding GABA_{A}-R Subunits.

GABA can enhance INS-1 cell proliferation (Soltani et al. (2011) Proc. Natl. Acad. Sci. USA, 108: 11692-11697), but it is unknown whether INS-1 cells express GABA_{A}-subunits that are sensitive to benzodiazepines. We tested INS-1 cell RNA for the expression of the GABA_{A}-R subunits that confer sensitivity to benzodiazepines (α₁, α₂, α₃, and α₅) using qRT-PCR. We detected α₁, α₃, and α₅ transcripts as well as β1, γ1, γ2, γ3 subunits (data not shown), indicating that INS-1 cells may express GABA_{A}-Rs that are sensitive to benzodiazepines.

INS-1 Cells Have Relatively Low Ability to Synthesize GABA.

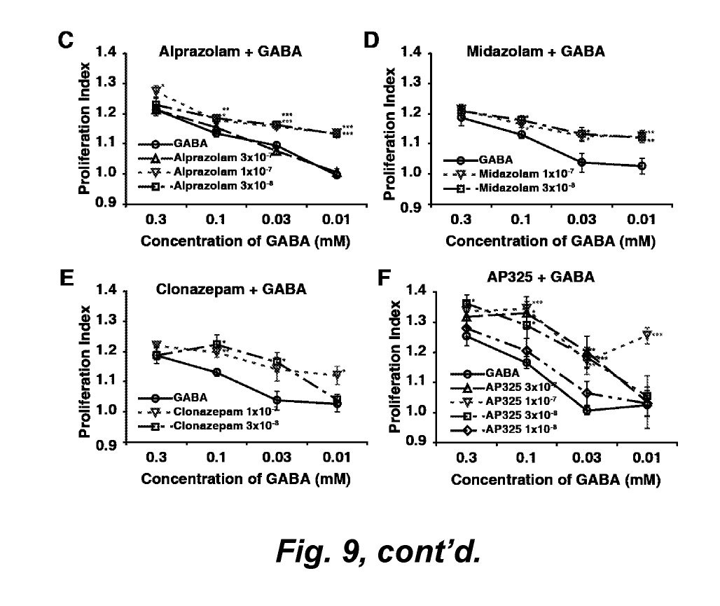

β-cells express GAD and secrete GABA which can act in an autocrine fashion. Whether INS-1 cells also synthesize GABA is unknown. We therefore tested GAD enzymatic activity in INS-1 cells. We found that GAD activity in INS-1 cells was considerably less than that in human islets, which in turn was less than that in mouse brain (FIG. 9, panel A). Nonetheless, GAD activity in INS-1 cells was consistently about 2-fold greater than that of negative control 293T cells, suggesting that they are capable of producing a low level of GABA that may act in an autocrine manner.

GABA_{A}-Rs PAMs Promote INS-1 Proliferation In Vitro.

We tested the impact of different concentrations of each PAM on the proliferation of INS-1 cells in vitro. Treatment with alprazolam or midazolam at 10 or/and 100 nM, but not with clonazepam or AP3 significantly stimulated proliferation of INS-1 cells (FIG. 9, panel B). Furthermore, treatment with GABA (alone) at 0.03-0.3 mM stimulated INS-1 cell proliferation (FIG. 9, panels C-G), similar to previous observations (Soltani et al. (2011) Proc. Natl. Acad. Sci. USA, 108: 11692-11697). While treatment with 0.01 mM GABA did not stimulate INS-1 proliferation (FIG. 9, panels C-G), the same dose of GABA in the presence of 30 or 100 nM alprazolam significantly increased INS-1 cell proliferation, which was significantly higher than that of alprazolam alone or even a 10-fold higher concentration of GABA alone (0.1 mM, FIG. 9, panel C). However, at a high concentration of GABA (0.1 or 0.3 mM), alprazolam had less ability to enhance GABA's pro-mitotic effects, perhaps because high concentrations of GABA already induced maximal GABA_{A}-R responses. Similarly, low doses of midazolam, clonazepam, or AP3 enhanced GABA-stimulated INS-1 cell proliferation (FIG. 9, panels D-G). Thus, GABA_{A}-R PAMs can potentiate GABA-induced β-cell proliferation.

Some benzodiazepines bind to the mitochondrial translocator protein (TSPO) which was previously referred to as a “peripheral benzodiazepine receptor”. Alprazolam does not bind to TSPO (Schmoutz et al. (2014) Behav. Brain. Res. 271: 269-276). To test whether midazolam and clonazepam may enhance INS-1 cell proliferation through the TSPO, INS-1 cells were cultured with the TSPO inhibitor PK11195 and stimulated with 0.3 mM GABA in the presence of midazolam or clonazepam. Treatment with PK1119 did not affect the ability of GABA and the tested benzodiazepines to enhance INS-1 cell replication (FIG. 9, panel H), indicating that this effect was not mediated through TSPO.

Alprazolam Enhances the Ability of Islet-Produced GABA to Promote Human Islet Cell Replication.

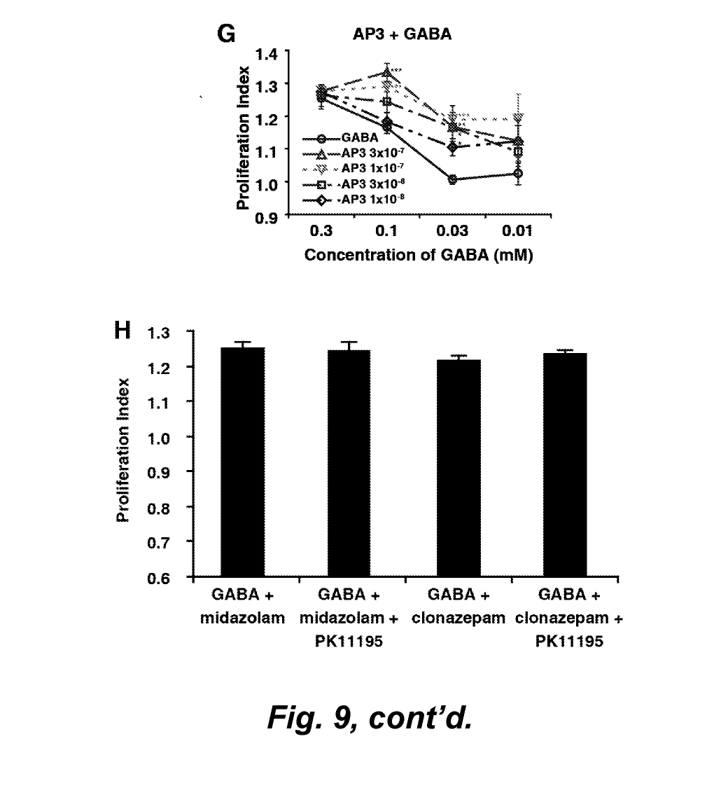

We next tested whether a PAM could enhance human islet cell replication. We focused on testing alprazolam because of its safety record and the finding that alprazolam treatment reduced HbA1c in diabetic patients (Lustman et al. (1995) Diabetes Care 18: 1133-1139). We cultured human islets with a dose range of alprazolam and observed that low concentrations of alprazolam promoted human islet cell proliferation (FIG. 10). The vast majority of replicating islet cells are likely to be β-cells because immunohistological studies have shown that GABA increases human β-cell replication but has no effect on α-cell replication (Purwana et al. (2014) Diabetes, 63(12): 4197-4205) and our unpublished observations), and other islet cell types do not express GABA-Rs. Addition of the Ca²⁺ channel blocker nifedipine (1 uM) to these cultures blocked the pro-mitotic effects of alprazolam (data not shown), consistent with the notion that this PAM enhances GABA_{A}-R-mediated activation of the PI3K/Akt pathway (et al. (2011) Proc. Natl. Acad. Sci. USA, 108: 11692-11697).

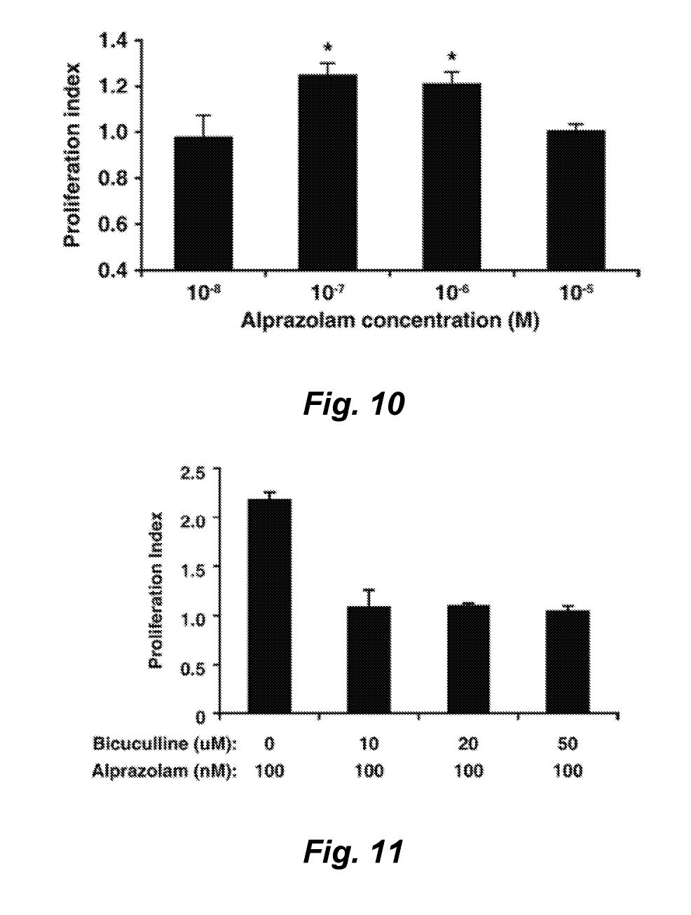

Since alprazolam is unable to significantly activate GABA_{A}-Rs independently of GABA, it likely that alprazolam acted in conjunction with GABA secreted from the islet β-cells to increase β-cell replication beyond its basal level. We added alprazolam to human islet cultures together with the GABA_{A}-R antagonist bicuculline which competitively inhibits GABA binding to GABA_{A}-Rs (Johnston (2013) Br. J. Pharmacol. 169: 328-336). We observed that bicuculline application ablated the ability of alprazolam to promote islet cell proliferation (FIG. 11). This suggests that islet-produced GABA may promote β-cell replication in an autocrine fashion and that enhancing GABA_{A}-R activity may push β-cell replication to a higher level.

The Combination of Alprazolam and GABA Has Enhanced Ability to Promote Human Islet Cell Replication at Low Dosage In Vitro.

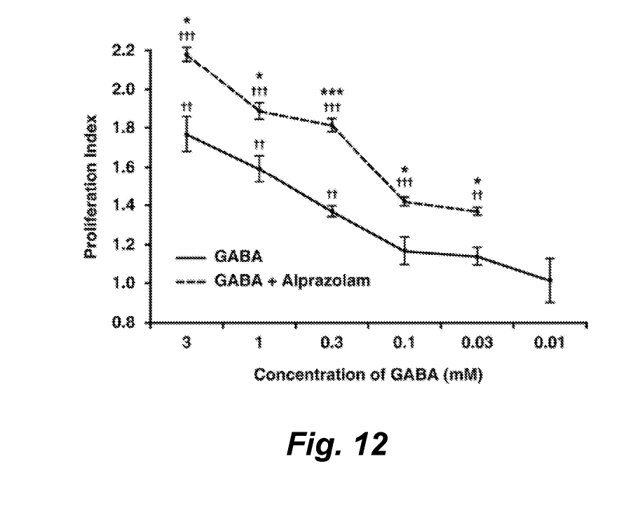

We next tested whether alprazolam could potentiate the ability of exogenous GABA to promote human islet cell replication in vitro. Human islets were treated with, or without, different concentrations of GABA in the presence or absence of 100 nM alprazolam. We found that GABA (alone) at 0.3-3 mM, but not a lower dose, significantly enhanced human islet cell proliferation in a dose-dependent manner (FIG. 12). Co-treatment with alprazolam and GABA significantly increased human islet cell proliferation relative to the corresponding GABA (alone) dose (FIG. 12). Notably, while treatment with 0.03 mM GABA did not significantly enhance human islet cell proliferation treatment, the same dose of GABA together with alprazolam significantly enhanced human islet cell proliferation (FIG. 12). To achieve a similar level of proliferation using GABA alone required a 10-fold higher level of GABA (0.3 mM). Thus, alprazolam greatly reduces the amount of GABA needed to induce β-cell proliferation.

### Discussion

GABA-R activation can protect β-cells from apoptosis and promote their replication (Ligon et al. (2007) Diabetologia, 50: 764-773; Soltani et al. (2011) Proc. Natl. Acad. Sci. USA, 108: 11692-11697; Tian et al. (2013) Diabetes, 62: 3760-3765; Prud'homme et al. (2103) Transplantation, 96(7): 616-623; Purwana et al. (2014) Diabetes, 63(12): 4197-4205). Oral GABA treatment increases human β-cell replication several fold, typically from less than 1% to about 2-3% of adult human β-cells in islet xenoplants (Tian et al. (2013) Diabetes, 62: 3760-3765; Purwana et al. (2014) Diabetes, 63(12): 4197-4205), which is similar to the maximum level of β-cell replication that takes place shortly after birth. This enhancement did not attenuate after five weeks of GABA treatment and led to increased β-cell mass and function in human islet xenoplants (Purwana et al. (2014) Diabetes, 63(12): 4197-4205). Accordingly, repurposing clinically applicable PAMs to enhance the activity of GABA produced within the islets, or the effect of exogenous GABA, is an attractive strategy for helping to treat T1D.

We first asked whether three clinically applicable BBB-permeable benzodiazepine PAMs, as well as a peripherally restricted non-benzodiazepine PAM, could promote INS-1 cell proliferation. We found that INS-1 cells express GABA_{A}-R subunits that should confer benzodiazepine sensitivity and that these cells could produce low levels of GABA. We observed that two of the four tested PAMs had a significant, albeit low, ability to promote INS-1 replication at at least one of the dosages tested. When GABA was exogenously provided to the culture media, all of the tested PAMs augmented the ability of low levels of GABA to induce INS-1 cell proliferation. Indeed, low levels of exogenous GABA that had little or no ability to promote INS-1 cell replication did so in the presence of nanomolar levels of each of the PAMs.

Of the four PAMs that we tested on INS-1 cells, we carried alprazolam's forward to study its effects on human islet cell replication because of alprazolam's safety profile and a previous observation that alprazolam treatment reduced HbA1c levels in diabetics ((Lustman et al. (1995) Diabetes Care 18: 1133-1139), see below). We observed that alprazolam (alone) enhanced human islet cell replication in vitro, most likely because it acted in conjunction with GABA secreted from β-cells. Indeed, blocking GABA binding with the antagonist bicuculline ablated the ability of alprazolam to enhance islet cell replication. This suggests that the GABA released from β-cells can have a pro-mitotic activity on β-cells and that this activity can be enhanced by a PAM. Addition of nifedipine to these cultures blocked alprazolam's pro-mitotic effects, suggesting it enhances GABA_{A}-R-mediated activation of the PI3K/Akt pathway (Soltani et al. (2011) Proc. Natl. Acad. Sci. USA, 108: 11692-11697).

In combination, alprazolam and exogenous GABA had a greater ability to promote human β-cell replication, and achieved a level of β-cell replication similar to that of GABA at ten-fold higher levels. The vast majority of islet cells that underwent replication in our assay are likely to be β-cells because immunohistological analyses have shown that GABA increases human β-cell replication but has no effect on α-cell replication (Purwana et al. (2014) Diabetes, 63(12): 4197-4205), and our unpublished observations), and islet δ and PP cells are not known to express GABA-Rs.

A clinical trial conducted in the early 1990's treated anxious and nonanxious individuals with poorly controlled diabetes with alprazolam to determine whether reducing anxiety could help anxious patients better manage their diabetes. The unexpected finding was that alprazolam treatment, but not placebo, reduced HbA1c levels regardless of whether the patients suffered from anxiety (Lustman et al. (1995) Diabetes Care 18: 1133-1139). These results were thought to stem from alprazolam's blunting of neurotransmitter and neurohormone release. In light of our findings, it is possible that alprazolam's beneficial effect on HbA1C may have arisen, at least in part, from enhancing the ability of islet GABA to promote β-cell survival and replication.

We envision several different paths by which our findings could lead to clinical benefits. First, it may be possible to improve β-cell mass and function in diabetics using BBB-permeable PAMs at doses below those used for CNS indications. Second, because the amount of β-cell mass following T1D onset is a major factor determining the success of interventive therapy, a short-term PAM treatment may help preserve residual β-cell mass and thereby improve the outcome of interventive therapies. Along this line, GABA treatment enhanced β-cell replication and survival in newly diabetic NOD mice (Soltani et al. (2011) Proc. Natl. Acad. Sci. USA, 108: 11692-11697; Tian et al. (2014) Diabetes, 63: 3128-3134), indicating that enhancing GABA-R activity can beneficial in the presence of β-cell autoreactivity even when little β-cell mass remains. Third, a short-term PAM treatment may help reduce β-cell loss due to hypoxia and stress following islet transplantation, as suggested by the ability of GABA treatment to improve β-cell survival in human islet xenografts (Tian et al. (2013) Diabetes, 62: 3760-3765; Purwana et al. (2014) Diabetes, 63(12): 4197-4205). Finally, immune cells also express GABA-Rs and their activation can inhibit proinflammatory immune responses such that GABA treatment ameliorates disease in mouse models of T1D, experimental autoimmune encephalomyelitis, rheumatoid arthritis and T2D (Soltani et al. (2011) Proc. Natl. Acad. Sci. USA, 108: 11692-11697; Prud'homme et al. (2103) Transplantation, 96(7): 616-623; Tian et al. (2014) Diabetes, 63: 3128-3134; Mendu et al. (2011) Mol. Immunol. 48: 399-407; Bhat et al. (2010) Proc. Natl. Acad. Sci. USA, 107: 2580-2585; Huang et al. (2015) J. Cell Physiol. 230: 1438-1447; Duthey et al. (2010) Exp. Dermatol. 19: 661-666; Tian et al. (2011) PLoS One, 6(9): e25338; Tian et al. (2014) J. Immunol. 173: 5298-5304; Tian et al. (2011) Autoimmunity, 44: 465-470). Accordingly, the use of peripherally-restricted GABA_{A}-R PAMs, or subclinical doses of BBB-permeable PAMs, in combination with low dose GABA may avoid CNS effects and promote β-cell mass/function as well as help control autoreactive T cell responses.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of promoting the replication, growth, and/or survival, and/or function of beta cells in mammal, said method comprising administering to said mammal a GABA_{A} receptor positive allosteric modulator (PAM) in an amount sufficient to promote the replication, and/or survival, and/or mass, and/or function of beta cells in said mammal.

2. A method of claim 1, wherein said GABA_{A} receptor positive allosteric modulator (PAM) is administered in conjunction with a GABA receptor activating ligand.

3. The method of claim 2, wherein the GABA receptor activating ligand when used in conjunction with said PAM is more effective to promote the replication, and/or survival, and/or function of beta cells in said mammal than when either agent is administered alone.

4. The method of claim 3, wherein the GABA receptor activating ligand when used in conjunction with a PAM is at least 10%, or at least 20%, or at least 30%, or at least 40%, or at least 50%, or at least 60%, or at least 70%, or at least 80%, or at least 90%, or at least 100%, or at least 1.2 fold, or at least 1.5 fold, or at least 2 fold, or at least 3 fold, or at least 4 fold, or at least 5 fold, or at least 10 fold is more effective to promote the replication, and/or survival, and/or function of beta cells in said mammal than when either agent is administered alone.

5. The method of claim 2, wherein the GABA receptor activating ligand is used at a lower dosage than would be used to achieve the same effect on beta cell replication, and/or survival, and/or function when used alone and/or said positive allosteric modulator is used at a lower dosage than would be used to achieve the activity for which the PAM is designed and/or approved if the PAM were used alone.

6. The method of claim 5, wherein the GABA receptor activating ligand used at less than about 95%, or less than about 90%, or less than about 80%, or less than about 70%, or less than about 60%, or less than about 50%, or less than about 40%, or less than about 30%, or less than about 20%, or less than about 10% of the dosage that would be used to achieve the same effect on beta cell replication, and/or survival, and/or function when the GABA receptor activating ligand is used alone.

7. The method according to any one of claims 5-6, wherein said positive allosteric modulator is used at less than about 95%, or less than about 90%, or less than about 80%, or less than about 70%, or less than about 60%, or less than about 50%, or less than about 40%, or less than about 30%, or less than about 20%, or less than about 10% of the dosage that that would be used to achieve the activity for which the PAM is designed and/or approved if the PAM were used alone.

8. The method according to any one of claims 5-7, where the GABA receptor activating ligand is used at a lower dosage than would be used to achieve the same effect on beta cell replication, and/or survival, and/or function when used alone and/or said positive allosteric modulator is used at a subtherapeutic dosage.

9. The method according to any one of claims 1-8, wherein said administration promotes replication of beta cells.

10. The method according to any one of claims 1-9, wherein said administration increases mass of beta cells.

11. The method according to any one of claims 1-10, wherein said administration promotes survival of beta cells.

12. The method according to any one of claims 1-11, wherein said administration promotes function of beta cells.

13. The method of claim 12, wherein said administration increases the insulin content and/or the amount of insulin secreted by beta cells.

14. The method of claim 12, wherein said administration promotes function by increasing the number of insulin positive beta cells.

15. The method according to any one of claims 1-14, wherein said mammal is a human.

16. The method of claim 15, wherein said mammal is a human diagnosed with type I diabetes.

17. The method of claim 15, wherein said mammal is diagnosed as pre-diabetic.

18. The method according to any one of claims 1-14, wherein said mammal is a non-human mammal.

19. The method according to any one of claims 1-18, wherein said GABA receptor activating ligand and said PAM act synergistically to improve the replication, and/or survival, and/or growth, and/or function of said beta cells.

20. The method according to any one of claims 1-19, wherein said PAM comprises an agent selected from the group consisting of AP325, and AP3.

21. The method of claim 20, wherein said PAM comprises AP325.

22. The method of claim 21, wherein said AP325 is administered at a dosage lower than that used for neuropathic pain or spinal cord injury.

23. The method according to any one of claims 1-19, wherein said PAM comprises an agent selected from the group consisting of a barbituate, a benzodiazepine, a quinazolinone, and a neurosteroid.

24. The method of claim 23, wherein said PAM comprises a barbituate.

25. The method of claim 24, wherein said PAM comprises a barbiturate selected from the group consisting of allobarbital (5,5-diallylbarbiturate), amobarbital (5-ethyl-5-isopentyl-barbiturate), aprobarbital (5-allyl-5-isopropyl-barbiturate), alphenal (5-allyl-5-phenyl-barbiturate), barbital (5,5-diethylbarbiturate), brallobarbital (5-allyl-5-(2-bromo-allyl)-barbiturate), pentobarbital (5-ethyl-5-(1-methylbutyl)-barbiturate), phenobarbital (5-ethyl-5-phenylbarbiturate), secobarbital (5-[(2R)-pentan-2-yl]-5-prop-2-enyl-barbiturate).

26. The method of claim 23, wherein said PAM comprises a benzodiazepine.

27. The method of claim 26, wherein said PAM comprises a benzodiazepine selected from the group consisting of alprazolam, bromazepam, chlordiazepoxide, midazolam, clonazepam, clorazepate, diazepam, estazolam, flurazepam, halazepam, ketazolam, lorazepam, nitrazepam, oxazepam, prazepam, quazepam, temazepam, and triazolam.

28. The method of claim 26, wherein said PAM comprises alprazolam.

29. The method of claim 28, wherein said alprazolam is administered at a dosage less than that used to treat used to treat an anxiety disorder, a panic disorder, and/or anxiety caused by depression.

30. The method of claim 28, wherein said alprazolam is administered as an immediate release tablet less than 1.5 mg orally per day or less than 1.0 mg orally per day, or less than 0.5 mg orally per day, or as an extended release tablet less than 0.5 mg orally per day, or less than about 0.4 mg orally per day or less than about 3 mg orally per day.

31. The method of claim 26, wherein said PAM comprises midazolam.

32. The method of claim 31, wherein said midazolam is administered at a dosage less than that used to reduce anxiety, or producing drowsiness or anesthesia before medical procedures or surgery, or to maintain sedation or anesthesia.

33. The method of claim 31, wherein said midazolam is administered at a dosage less than 1 mg IV, or less than about 0.8 mg IV, or less than about 0.5 mg IV, or less than about 0.01 mg/kg IV, or less than about 0.07 mg/kg IM, or less than about 0.05 mg/kg IM, or less than about 0.03 mg/kg IM, or less than about 0.01 mg/kg.

34. The method of claim 26, wherein said PAM comprises clonazepam.

35. The method of claim 34, wherein said clonazepam is administered at a dosage less than that used to treat seizure disorders (including absence seizures or Lennox-Gastaut syndrome), or less than that used to treat panic disorder (including agoraphobia) in adults.

36. The method of claim 34, wherein said clonazepam is administered at a dosage less than about 0.5 mg orally per day, or less than about 0.25 mg orally per day, or less than bout 0.01 mg/kg/day, or less than about 0.005 mg/kg/day.

37. The method of claim 23, wherein said PAM comprises a neurosteroid.

38. The method of claim 37, wherein said PAM comprises a neurosteroid selected from the group consisting of allopregnanolone (3α-hydroxy-5α-pregnan-20-one), and pregnanolone.

39. The method according to any one of claims 2-38, wherein said GABA receptor activating ligand comprises GABA.

40. The method according to any one of claims 2-38, wherein said GABA receptor activating ligand comprises an agent selected from the group consisting of bamaluzole, gabamide, GABOB, gaboxadol, ibotenic acid, isoguvacine, isonipecotic acid, muscimol, phenibut, picamilon, progabide, quisqualamine, progabide acid (SL 75102), thiomuscimol, pregabalin, vigabatrin, 6-aminonicotinic acid, homotaurine, and XP13512 [(±)-1-([(α-isobutanoyloxyethoxy) carbonyl] aminomethyl)-1-cyclohexane acetic acid].

41. The method according to any one of claims 1-40, wherein said method increases beta cell survival after islet transplantation.

42. The method according to any one of claims 1-41, wherein said method increases beta cell replication in islets after islet transplantation.

43. The method according to any one of claims 1-40, wherein said method is performed after islet implantation in order to reduce β-cell loss due to hypoxia and stress.

44. The method according to any one of claims 1-43, where said method is performed for up to 3 days post implantation, or for up to 1 week post implantation, or for up to 2 weeks post implantation, or for up to 3 weeks post implantation, or for up to 4 weeks post implantation, or for up to 5 weeks post implantation, or for up to 6 weeks post implantation, or for up to 7 weeks post implantation, or for up to 8 weeks post implantation, or for up to 3 months post implantation, or for up to 4 months post implantation, or for up to 5 months post implantation, or for up to 6 months post implantation.

45. The method according to any one of claims 2-44, wherein said GABA receptor activating ligand is not an alcohol.

46. The method according to any one of claims 2-45, wherein said GABA receptor activating ligand is not a kavalactone.

47. The method according to any one of claims 2-46, wherein said GABA receptor activating ligand is not skullcap or a skullcap constituent.

48. The method according to any one of claims 2-47, wherein said GABA receptor activating ligand is not valerian or a valerian constituent.

49. The method according to any one of claims 2-48, wherein said GABA receptor activating ligand is not a volatile gas.

50. The method according to any one of claims 1-49, wherein said mammal is not under treatment for one or more conditions selected from the group consisting of neuropathic pain, spinal cord injury, an anxiety disorder, a panic disorder, anxiety caused by depression, a seizure disorder (including absence seizures or Lennox-Gastaut syndrome), and catamenial epilepsy.

51. The method according to any one of claims 1-50, wherein said PAM is not administered to produce drowsiness or anesthesia before a medical procedure or surgery, or to maintain sedation or anesthesia.

52. The method according to any one of claims 1-51, wherein said PAMS that are BBB-permeable are administered at doses below those used for CNS indications.

53. A pharmaceutical formulation comprising a GABA receptor activating ligand and a GABA_{A} receptor positive allosteric modulator (PAM).

54. The formulation of claim 53, wherein the GABA receptor activating ligand is present at a lower unit dosage than in a therapeutic formulation comprising a GABA receptor activating ligand alone and/or the PAM is at a lower unit dosage than in a therapeutic formulation comprising the PAM alone.

55. The formulation of claim 54, wherein the GABA receptor activating ligand is present at a lower unit dosage than in a therapeutic formulation comprising GABA or a GABA analog alone.

56. The formulation of claim 55, wherein the GABA receptor activating ligand present at less than about 95%, or less than about 90%, or less than about 80%, or less than about 70%, or less than about 60%, or less than about 50%, or less than about 40%, or less than about 30%, or less than about 20%, or less than about 10% of the dosage that would be provided to achieve the same effect on beta cell replication, and/or survival, and/or function when the GABA receptor activating ligand is present alone.

57. The formulation according to any one of claims of claim 53-56, wherein the PAM is at a lower unit dosage than in a therapeutic formulation comprising the PAM alone.

58. The formulation of claim 57, wherein said positive allosteric modulator is present at less than about 95%, or less than about 90%, or less than about 80%, or less than about 70%, or less than about 60%, or less than about 50%, or less than about 40%, or less than about 30%, or less than about 20%, or less than about 10% of the amount that that would be present to achieve the activity for which the PAM is designed and/or approved if the PAM were used alone.

59. The formulation according to any one of claims 53-58, wherein said GABA or GABA analog and said PAM are present in a concentration sufficient to provide synergistic activity in stimulating replication of cells expressing a GABA_{A} receptor.

60. The formulation according to any one of claims 53-58, wherein said GABA receptor activating ligand and said PAM are present in a concentration sufficient to provide synergistic activity in stimulating replication of beta cells and/or promoting survival of beta cells and/or improving function of beta cells.

61. The formulation according to any one of claims 53-60, wherein said GABA receptor activating ligand and said PAM are present in a concentration sufficient provide at least 10%, or at least 20%, or at least 30%, or at least 40%, or at least 50%, or at least 60%, or at least 70%, or at least 80%, or at least 90%, or at least 100%, or at least 1.2 fold, or at least 1.5 fold, or at least 2 fold, or at least 3 fold, or at least 4 fold, or at least 5 fold, or at least 10 fold greater efficacy in promoting the replication, and/or survival, and/or function of beta cells in a mammal than when either agent is administered alone.

62. The formulation according to any one of claims 53-61, wherein said PAM comprises an agent selected from the group consisting of a barbituate, a benzodiazepine, a quinazolinone, and a neurosteroid.

63. The formulation of claim 62, wherein said PAM comprises a barbituate.

64. The formulation of claim 63, wherein said PAM comprises a barbiturate selected from the group consisting of allobarbital (5,5-diallylbarbiturate), amobarbital (5-ethyl-5-isopentyl-barbiturate), aprobarbital (5-allyl-5-isopropyl-barbiturate), alphenal (5-allyl-5-phenyl-barbiturate), barbital (5,5-diethylbarbiturate), brallobarbital (5-allyl-5-(2-bromo-allyl)-barbiturate), pentobarbital (5-ethyl-5-(1-methylbutyl)-barbiturate), phenobarbital (5-ethyl-5-phenylbarbiturate), secobarbital (5-[(2R)-pentan-2-yl]-5-prop-2-enyl-barbiturate).

65. The formulation of claim 62, wherein said PAM comprises a benzodiazepine.

66. The formulation of claim 65, wherein said PAM comprises a benzodiazepine selected from the group consisting of alprazolam, bromazepam, chlordiazepoxide, midazolam, clonazepam, clorazepate, diazepam, estazolam, flurazepam, halazepam, ketazolam, lorazepam, nitrazepam, oxazepam, prazepam, quazepam, temazepam, and triazolam.

67. The formulation of claim 65, wherein said PAM comprises alprazolam.

68. The formulation of claim 65, wherein said PAM comprises midazolam.

69. The formulation of claim 65, wherein said PAM comprises clonazepam.

70. The formulation of claim 62, wherein said PAM comprises a neurosteroid.

71. The formulation of claim 70, wherein said PAM comprises a neurosteroid selected from the group consisting of allopregnanolone, and pregnanolone.

72. The formulation according to any one of claims 53-71, wherein said GABA receptor activating ligand comprises GABA.

73. The formulation according to any one of claims 53-71, wherein said GABA receptor activating ligand an agent selected from the group consisting of bamaluzole, gabamide, GABOB, gaboxadol, ibotenic acid, isoguvacine, isonipecotic acid, muscimol, phenibut, picamilon, progabide, quisqualamine, progabide acid (SL 75102), thiomuscimol, pregabalin, vigabatrin, 6-aminonicotinic acid, homotaurine, and XP13512 [(±)-1-([(α-isobutanoyloxyethoxy) carbonyl] aminomethyl)-1-cyclohexane acetic acid].

74. The formulation according to any one of claims 53-73, wherein said GABA receptor activating ligand is not an alcohol.

75. The formulation according to any one of claims 53-74, wherein said GABA receptor activating ligand is not a kavalactone.

76. The formulation according to any one of claims 53-75, wherein said GABA receptor activating ligand is not skullcap or a skullcap constituent.

77. The formulation according to any one of claims 53-76, wherein said GABA receptor activating ligand is not valerian or a valerian constituent.

78. A kit for promoting the replication in a mammal of cells that express GABA_{A} receptors, said kit comprising:
a container containing a GABA receptor activating ligand; and
a container containing a GABA_{A} receptor positive allosteric modulator (PAM).

79. The kit of claim 78 where the GABA receptor activating ligand and the PAM are in the same container.

80. The kit of claim 78 where the GABA receptor activating ligand and the PAM are in separate containers.

81. The kit according to any one of claims 78-80, wherein the GABA receptor activating ligand is present at a lower unit dosage than in a therapeutic formulation comprising GABA or a GABA analog alone and/or the PAM is at a lower unit dosage than in a therapeutic formulation comprising the PAM alone.

82. The kit according to any one of claims 78-80, wherein the GABA receptor activating ligand is present at a lower unit dosage than in a therapeutic formulation comprising GABA or a GABA analog alone.

83. The kit of claim 82, wherein the GABA receptor activating ligand present at less than about 95%, or less than about 90%, or less than about 80%, or less than about 70%, or less than about 60%, or less than about 50%, or less than about 40%, or less than about 30%, or less than about 20%, or less than about 10% of the dosage that would be provided to achieve the same effect on beta cell replication, and/or survival, and/or function when the GABA receptor activating ligand is present alone.

84. The kit according to any one of claims 78-83, wherein the PAM is at a lower unit dosage than in a therapeutic formulation comprising the PAM alone.

85. The kit according to any one of claims 78-84 wherein said positive allosteric modulator is present at less than about 95%, or less than about 90%, or less than about 80%, or less than about 70%, or less than about 60%, or less than about 50%, or less than about 40%, or less than about 30%, or less than about 20%, or less than about 10% of the amount that that would be present to achieve the activity for which the PAM is designed and/or approved if the PAM were used alone.

86. The kit according to any one of claims 78-85, wherein said GABA receptor activating ligand and said PAM are present in a concentration sufficient to provide synergistic activity in stimulating replication of cells expressing a GABA_{A} receptor.

87. The kit of according to any one of claims 78-85, wherein said GABA receptor activating ligand and said PAM are present in a concentration sufficient to provide synergistic activity in stimulating replication of beta cells and/or promoting survival of beta cells and/or improving function of beta cells.

88. The kit according to any one of claims 78-87, wherein said GABA receptor activating ligand and said PAM are present in a concentration sufficient provide at least 10%, or at least 20%, or at least 30%, or at least 40%, or at least 50%, or at least 60%, or at least 70%, or at least 80%, or at least 90%, or at least 100%, or at least 1.2 fold, or at least 1.5 fold, or at least 2 fold, or at least 3 fold, or at least 4 fold, or at least 5 fold, or at least 10 fold greater efficacy in promoting the replication, and/or survival, and/or function of beta cells in a mammal than when either agent is administered alone.

89. The kit according to any one of claims 78-88, wherein said PAM comprises an agent selected from the group consisting of a barbituate, a benzodiazepine, a quinazolinone, and a neurosteroid.

90. The kit of claim 89, wherein said PAM comprises a barbituate.

91. The kit of claim 90, wherein said PAM comprises a barbiturate selected from the group consisting of allobarbital (5,5-diallylbarbiturate), amobarbital (5-ethyl-5-isopentyl-barbiturate), aprobarbital (5-allyl-5-isopropyl-barbiturate), alphenal (5-allyl-5-phenyl-barbiturate), barbital (5,5-diethylbarbiturate), brallobarbital (5-allyl-5-(2-bromo-allyl)-barbiturate), pentobarbital (5-ethyl-5-(1-methylbutyl)-barbiturate), phenobarbital (5-ethyl-5-phenylbarbiturate), secobarbital (5-[(2R)-pentan-2-yl]-5-prop-2-enyl-barbiturate).

92. The kit of claim 89, wherein said PAM comprises a benzodiazepine.

93. The kit of claim 92, wherein said PAM comprises a benzodiazepine selected from the group consisting of alprazolam, bromazepam, chlordiazepoxide, midazolam, clonazepam, clorazepate, diazepam, estazolam, flurazepam, halazepam, ketazolam, lorazepam, nitrazepam, oxazepam, prazepam, quazepam, temazepam, and triazolam.

94. The kit of claim 92, wherein said PAM comprises alprazolam.

95. The kit of claim 92, wherein said PAM comprises clonazepam.

96. The kit of claim 92, wherein said PAM comprises midazolam.

97. The kit of claim 89, wherein said PAM comprises a neurosteroid.

98. The kit of claim 97, wherein said PAM comprises a neurosteroid selected from the group consisting of allopregnanolone, and pregnanolone.

99. The kit according to any one of claims 78-98, wherein said GABA receptor activating ligand comprises GABA.

100. The kit according to any one of claims 78-98, wherein said GABA receptor activating ligand comprises an agent selected from the group consisting of bamaluzole, gabamide, GABOB, gaboxadol, ibotenic acid, isoguvacine, isonipecotic acid, muscimol, phenibut, picamilon, progabide, quisqualamine, progabide acid (SL 75102), thiomuscimol, pregabalin, vigabatrin, 6-aminonicotinic acid, homotaurine, and XP13512 [(±)-1-([(α-isobutanoyloxyethoxy) carbonyl] aminomethyl)-1-cyclohexane acetic acid].

101. The kit according to any one of claims 78-100, wherein said GABA receptor activating ligand is not an alcohol.

102. The kit according to any one of claims 78-101, wherein said GABA receptor activating ligand is not a kavalactone.

103. The kit according to any one of claims 78-102, wherein said GABA receptor activating ligand is not skullcap or a skullcap constituent.

104. The kit according to any one of claims 78-103, wherein said GABA receptor activating ligand is not valerian or a valerian constituent.
